SEQUENCE LISTING (10 sequences)

SEQ ID NO: 1 - protein, 227 aa, Homo sapiens
MAPYHIRKYQESDRKSVVGLLSRGMAEHAPATFRRLLKLPRTLILLLGGALALLLVSGSWILALVFSLSLLPALWFLAKKPWTRYVDIALRTDMSDITKSYLSECGSCFWVGESEEKVVGTVGALPVDDPTLREKRLQLFHLSVDNEHRGQGIAKALVRTVLQFARDQGYSEVVLDTSNIQLSAMGLYQSLGFKKTGQSFFHVWARLVDLHTVHFIYHLPSAQAGRL

SEQ ID NO: 2 - protein, 227 aa, Homo sapiens
MAPCHIRKYQESDRQWVVGLLSRGMAEHAPATFRQLLKLPRTLILLLGGPLALLLVSGSWLLALVFSISLFPALWFLAKKPWTEYVDMTLCTDMSDITKSYLSERGSCFWVAESEEKVVGMVGALPVDDPTLREKRLQLFHLFVDSEHRRQGIAKALVRTVLQFARDQGYSEVILDTGTIQLSAMALYQSMGFKKTGQSFFCVWARLVALHTVHFIYHLPSSKVGSL

SEQ ID NO: 3 - DNA, 684 bp, Homo sapiens
atggctccttatcacatccgcaaataccaggagagcgaccgcaagtcggtcgtgggcttgctctccggggggatggccgaacacgccccagccaccttccggcgattactgaagctgcctcgaaccctcatactcttacttgggggggcccttgccctactcctggtctctggctcctggattctggccctcgtgttcagcctcagcctccttcctgccctgtggttccttgccaaaaaaccctggacgcggtatgtagacatagcattgcgcacagacatgtctgacatcaccaaatcctacctgagtgagtgtggctcctgcttctgggtggctgaatctgaagagaaggtggtgggcacagtaggagctctgcccgttgatgatcccaccttgagggagaagcggttgcagctgtttcatctctctgtggacaatgagcaccgtggtcaggggatagcaaaagccctggtcaggactgtcctccagtttgcccgggaccagggctacagtgaagttgtcctggacaccagcaacatccagctctctgccatgggcctctaccagagcttgggcttcaagaagacgggccagtccttcttccacgtgtgggccaggctggtggatcttcatacagttcatttcatctatcacctcccttctgctcaggcagggcgtctatga

SEQ ID NO: 4 - DNA, 684 bp, Homo sapiens
atggctccttgtcacatccgcaaataccaggagagcgaccgccagtgggttgtgggcttgctctcccgggggatggccgagcatgccccagccaccttccggcaattgctgaagctgcctcgaaccctcatactcttacttggggggcccctcgccctactcctggtctctggatcctggcttctagccctcgtgttcagcatcagcctcttccctgccctgtggttccttgccaaaaaaccctggacggagtatgtggacatgacattgtgcacagacatgtctgacattaccaaatcctacctgagtgagcgtggctcctgcttctgggtggctgagtctgaagagaaggtggtgggcatggtaggagctctgcctgttgatgatcccaccttgagggagaagcggttgcagctgtttcatctctttgtggacagtgagcaccgtcgtcaggggatagcaaaagccctggtcaggactgtcctccagtttgcccgggaccagggctacagtgaagttatcctggacaccggcaccatccagctctctgctatggccctctaccagagcatgggcttcaagaagacgggccagtccttcttctgtgtgtgggccaggctagtggctcttcatacagttcatttcatctaccacctcccttcttctaaggtagggagtctgtga

SEQ ID NO: 5 - DNA, 19 bp, Artificial
  GAPDH forward primer
gaaggtgaaggtcggagtc

SEQ ID NO: 6 - DNA, 20 bp, Artificial
  GAPDH reverse primer
gaagatggtgatgggatttc

SEQ ID NO: 7 - DNA, 21 bp, Artificial
  ATase1 forward primer
cgattactgaagctgcctcga

SEQ ID NO: 8 - DNA, 21 bp, Artificial
  ATase1 reverse primer
ggttttttggcaaggaaccac

SEQ ID NO: 9 - DNA, 20 bp, Artificial
  ATase2 forward primer
tccttgccaaaaaaccctgg

SEQ ID NO: 10 - DNA, 21 bp, Artificial
  ATase2 reverse primer
atgcccaccaccttctcttca

(19) United States
(12) Patent Application Publication
(10) Pub. No.: US 2009/0136977 A1
(43) Pub. Date: May 28, 2009

(54) NOVEL TARGETS FOR ALZHEIMER'S DISEASE

(76) Inventor: Luigi Puglielli, Madison, WI (US)

Correspondence Address: WARF/BHGL, P.O. Box 10395, Chicago, IL, 60610, US

(21) Appl. No.: 12/263,138

(22) Filed: Oct. 31, 2008

Related U.S. Application Data
(60) Provisional application No. 60/985,361, filed on Nov. 5, 2007.

(51) Int. Cl. C12Q 1/48; C12N 9/10

(52) U.S. Cl. 435/15; 435/193

(57) ABSTRACT

Compositions and methods for post-translational modifications that include acetylCoA:lysine acetyltransferase activity in the ER lumen are provided. The disclosed compositions and methods are especially suited for the identification of compounds useful for the prevention or treatment of neurodegenerative diseases such as Alzheimer's.

### CROSS-REFERENCE TO RELATED APPLICATIONS

This invention claims priority to U.S. Provisional Patent Application Ser. No. 60/985,361, filed Nov. 5, 2007, which is herein incorporated by reference.

### GOVERNMENT INTERESTS

This invention was made with United States government support awarded by the National Institutes of Health, Grant No. NS 045669. The United States government has certain rights in this invention.

### FIELD OF THE INVENTION

The present invention relates generally to pharmacological targets for neurological diseases. More particularly, the present invention relates to pharmacological targets for Alzheimer's disease.

### BACKGROUND

The efficiency of folding, conformational maturation, and molecular stability of nascent membrane and secretory proteins is greatly affected in the endoplasmic reticulum (ER) by post-translational events that modify, either temporally or definitively, the protein (Trombetta and Parodi, 2003, Annu. Rev. Cell Dev. Biol. 19: 649-676). One of the best characterized forms of transient modification is the attachment of a glucose residue to improperly folded nascent glycoproteins by the UDP-glucose: glycoprotein glucosyltransferase. This transient form of glucosylation regulates the interaction of the nascent protein with the chaperone calnexin and its ability to leave the early secretory pathway. The mechanism works in a way that successfully folded proteins dissociate from the calnexin/calreticulin cycle and advance toward the Golgi apparatus, whereas misfolded intermediates are directed toward the ER-associated degradation (ERAD) system.

A novel form of post-translational regulation of the β-site amyloid precursor protein (APP) cleaving enzyme 1 (BACE1) was recently identified (Costantini et al., 2007, Biochem. J. 407: 383-395). BACE1 is a membrane protein that acts as the rate-limiting enzyme in the generation of the Alzheimer's disease amyloid β-peptide (Aβ) from the APP. Specifically, nascent BACE1 is transiently acetylated in seven lysine residues clustered in a highly-disordered region of the protein that faces the lumen of the ER. The lysine acetylation of nascent BACE1 regulates its ability to achieve conformational maturation/stability and to advance toward the Golgi apparatus, where a Golgi-based deacetylase removes the acetyl groups. Non-acetylated, presumably misfolded, intermediates of the nascent protein are retained in the early secretory pathway and degraded by PCSK9/NARC-1 (Jonas et al., 2008, EMBO Rep. 9: 916-922). The acetylation of nascent BACE1 in the lumen of the ER and/or ER Golgi Intermediate Compartment (ERGIC) requires a membrane transporter that translocates acetylCoA (acetyl coenzyme A), the donor of the acetyl group, from the cytoplasm to the lumen of the ER and one or more ER/ERGIC-based acetyCoA:lysine acetyltransferases (ATases).

The acetylation/deacetylation process is tightly regulated by the lipid second messenger ceramide and is under the control of the general aging program mediated by the insulin-like growth factor 1 receptor (IGF1-R) (Costantini et al., 2006, EMBO J. 25: 1997-2006; Puglielli, 2008, Neurobiol. Aging 29: 795-811). Ceramide, the last output of the above pathway, regulates the efficiency of acetylCoA translocation into the ER lumen and the rate of deacetylation in the Golgi apparatus. The ER-based acetyltransferase (or acetyltransferases) appear to be regulated by the availability of the donor, most likely through an allosteric mechanism.

### BRIEF SUMMARY

Methods are provided, which include: a) reacting polypeptides at least 87% identical to the amino acid sequence of SEQ ID NO:1 or SEQ ID NO:2, substrates, ER-derived vesicles, and an acetylCoA, and b) measuring the acetyltransferase activity of the polypeptides, where the acetyltransferase activity comprises acetylation of the substrate. The polypeptides may be at least 95% identical to the amino acid sequence of SEQ ID NO:1. Alternatively, the polypeptides may be at least 95% identical to the amino acid sequence of SEQ ID NO:2. The methods may include measuring the acetylCoA:lysine acetyltransferase activity of the polypeptides. The methods may include measuring the acetyltransferase activity in the ER-derived vesicles. The substrates may be labeled. The substrates may be BACE1. The polypeptides may be purified, isolated, or recombinant polypeptides. In the practice of the methods, acetylation may include acetylation of one or more lysine residues of the substrate. The methods may be carried out in solution, on solid support, or both in solution and on solid support.

Provided are in vitro methods for identification of candidate compounds as compounds that may be useful for the treatment of Alzheimer's disease. The methods include the steps of: a) providing polypeptides at least 87% identical to the amino acid sequence of SEQ ID NO:1 or SEQ ID NO:2, b) contacting the polypeptides with the candidate compounds, and c) measuring the acetyltransferase activity of the polypeptides, where a decrease in the acetyltransferase activity of the polypeptides, relative to the acetyltransferase activity of the polypeptides not contacted with the candidate compounds, identifies the candidate compounds as compounds that may be useful for the treatment of Alzheimer's disease. The polypeptides may acetylate an aspartic peptidase in the ER. The aspartic peptidase may be labeled. The aspartic peptidase may be BACE1. The polypeptides may be ATase1. Alternatively, the polypeptides may be ATase2. The methods may include measuring the acetylCoA:lysine acetyltransferase activity of the polypeptides. The methods may be carried out in solution, on solid support, or both in solution and on solid support. The polypeptides may be expressed in cells.

Provided are in vitro methods, which include: reacting ATase1 and/or ATase2 with a known concentration of substrate and a known concentration of test compounds; and measuring the acetyltransferase activity of the ATase1 and/or ATase2. A decrease in the acetyltransferase activity of the ATase1 and/or ATase2, relative to the acetyltransferase activity of the ATase1 and/or ATase2 in the absence of the candidate compounds, identifies the candidate compounds as compounds that may be useful for the inhibition of the acetyltransferase activity of the ATase1 or ATase2. The acetyltransferase activity may be measured in an endoplasmic reticulum. The substrate may be labeled. The substrate may be BACE1. The methods may include measuring the acetylCoA:lysine acetyltransferase activity of the ATase1 and/or ATase2. The methods may be carried out in solution, on solid support, or both in solution and on solid support.

Provided are isolated or recombinantly produced polypeptides having an amino acid sequence at least 87% identical with an amino acid sequence selected from a group consisting of the amino acid sequence of SEQ ID NO:1 and SEQ ID NO:2, where the polypeptides are capable of acetylating substrates. The polypeptides may be capable of acetylating substrates in an endoplasmic reticulum. The polypeptides may have acetylCoA:lysine acetyltransferase activity. The polypeptides may be capable of acetylating BACE1.

Provided are methods, which include acetylating a substrate using isolated or recombinantly produced polypeptides having an amino acid sequence at least 87% identical with an amino acid sequence selected from a group consisting of the amino acid sequence of SEQ ID NO:1 and SEQ ID NO:2. The substrate may be BACE1.

Provided is a system, which includes: a) an isolated polypeptide having an amino acid sequence at least 87% identical with an amino acid sequence selected from a group consisting of the amino acid sequence of SEQ ID NO:1 and SEQ ID NO:2, b) an acetyl donor, and c) a substrate, where the polypeptide has an acetyltransferase activity for acetylating the substrate. In the system, the polypeptide may have acetylCoA:lysine acetyltransferase activity. The acetyl donor may be acetyl coenzyme A. The substrate may be BACE1.

### BRIEF DESCRIPTION OF THE DRAWINGS

FIG. 1 shows an alignment (A) of the amino acid sequences of ATase1 (acetyltransferase 1) and ATase2 (acetyltransferase 2), and a schematic plot (B) showing a hydrophobicity view of ATase1 and ATase2.

FIG. 2 shows images (A, C, D) and graphs (B, D, E) illustrating how ATase1 is localized in the early secretory pathway and has acetylCoA:lysine acetyltransferase activity.

FIG. 3 shows a graph (A) and images (B) illustrating how ATase1 acetylates BACE1 in vitro.

FIG. 4 shows images (A, C) and graphs (B, D) illustrating how ATase2 is localized in the early secretory pathway and has acetylCoA:lysine acetyltransferase activity.

FIG. 5 shows images (A) and graphs (B, C) illustrating how ATase2 acetylates BACE1 in vitro.

FIG. 6 shows images (A, B, D, F, H) and graphs (C, E, G, I) illustrating how ATase1 and ATase2 regulate the steady-state levels of and the generation of Aβ.

FIG. 7 shows graphs (A, B) illustrating how ATase1 and ATase2 are upregulated in AD brains and following ceramide treatment.

FIG. 8 shows graphs (A, B) and an image (C) illustrating that the C-terminal and catalytically active domain of ATase1 and ATase2 faces the luminal site of the ER/ERGIC system.

FIG. 9 shows images (A) and a schematic diagram (B) illustrating the subcellular distribution profile of BACE1, ATase1, and ATase2.

### DETAILED DESCRIPTION OF THE PRESENTLY PREFERRED EMBODIMENTS

The practice of the present invention employs, unless otherwise indicated, conventional techniques of protein chemistry and synthesis, molecular biology (including recombinant techniques), microbiology, cell biology, biochemistry, immunology, protein kinetics, and mass spectroscopy, which are within the skill of art. Such techniques are explained fully in the literature, e.g. in Sambrook et al., 2000, Molecular Cloning: A Laboratory Manual, 3rd ed., Cold Spring Harbor Laboratory Press; Current Protocols in Molecular Biology Volumes 1-3, John Wiley & Sons, Inc.; Kriegler, 1990, Gene Transfer and Expression: A Laboratory Manual, Stockton Press, New York; Dieffenbach et al., 1995, PCR Primer: A Laboratory Manual, Cold Spring Harbor Laboratory Press, each of which is incorporated herein by reference in its entirety.

Generally, the nomenclature and the laboratory procedures in recombinant DNA technology described below are those well known and commonly employed in the art. Standard techniques are used for cloning, DNA and RNA isolation, amplification and purification. Generally, enzymatic reactions involving DNA ligase, DNA polymerase, restriction endonucleases and the like are performed according to the manufacturer's specifications. Procedures employing commercially available assay kits and reagents are typically used according to manufacturer-defined protocols unless otherwise noted.

“Acetylation” (or in International Union of Pure and Applied Chemistry-IUPAC nomenclature “ethanoylation”) describes a reaction that introduces an acetyl functional group into an organic compound, for example protein. For example, acetylation of a NH₂ in lysine yields an amide (NCOCH₃); acetylation of a hydroxyl group yields an ester (OCOCH₃). Deacetylation is the removal of the acetyl group. Acetylation occurs as one type of co-translational or post-translational modification of proteins, such as histones and tubulins. Acetylation may occur on particular amino acid residues. An example of that is the reversible acetylation of lysine residues in histones and transcription factors.

“Post-translational modification” refers to the structural modifications of proteins after they have been translated. It is one of the later steps in protein biosynthesis for many proteins. The post-translational modifications can be in the form of covalent modifications or non-covalent modifications. Examples of covalent post-translational modifications include signal peptide cleavage, phosphorylation, acetylation, adenylation, proteolysis, amino peptidase clipping, arginylation, disulphide bond formation and cleavage, amidation, glycosylation, isoprenylation, myristoylation, ubiquitination, SUMOlation, and covalent addition of proteins including Agp12 and Nedd8. Examples of non-covalent post-translational modifications include changes in the structure or folding state of a given protein (e.g., denaturation) or its non-covalent interactions with other proteins, nucleic acid, carbohydrate, drugs, compounds or lipids. Some modifications, like phosphorylation, are part of common mechanisms for controlling the behavior of a protein, for instance activating or inactivating an enzyme. Other examples include the binding of a protein as a homo or hetero polymer, insertion in to a lipid membrane, or binding to a glycosyl group, or binding to a drug or compound.

In vitro translation systems may introduce post-translational modifications into translated proteins. Often, these post-translational modifications may be indicative of post-translational modifications that are observed in vivo. Advantageously, the methods of the invention may be used to identify proteins that receive specific types of post-translational modifications. The methods of the present invention are particularly suited for the identification of proteins that are acetylated in the ER, and deacetylated in the Golgi apparatus.

“Protein translocation” or “translocation”, as used herein, refers to the translocation of proteins across biological membranes (reviewed, e.g., in Wickner and Schekman, 2005, Science 310: 1452-1456). Eukaryotic proteins are synthesized within the endoplasmic reticulum (ER), are delivered from the ER to the Golgi apparatus (also known as Golgi complex) for post-translational processing and sorting, and are transported from the Golgi to specific intracellular and extracellular destinations. Translocation thus includes transport of proteins from the ER to the Golgi apparatus.

In one aspect, the present invention provides applications such as, for example, assays for enzymatic activity, and assays for substrate activity. These assay may in particular measure the acetyltransferase activity of ATase1, ATase2, or both ATase1 and ATase2. In such assays, the acetyltransferase activity of ATase1 and/or ATase2 may be measured in a variety of ways known in the art. The acetyltransferase(s) may be immobilized to solid support prior to conducting of the assays. In other applications, it may be desirable to contact the acetyltransferase(s) with a modifying reagent to introduce modifications prior to conducting an assay. As well, in other applications, it may be desirable to contact the acetyltransferase(s) with a candidate test compound (e.g., candidate inhibitor, agonist, or promoter), prior to conducting an assay, or during conducting the assay. This technique enables the screening of a variety of compounds, candidate inhibitors, agonists, or promoters, etc., and analysis of the effect of one or more of these on the acetyltransferase activity of ATase1, ATase2, or both ATase1 and ATase2.

The invention relates, in part, to the production and analysis of proteins formed by the in vitro transcription and translation of nucleic acid constructs. These nucleic acid constructs are characterized by their ability to direct an RNA polymerase to produce an RNA transcript that, in turn, is able to direct the synthesis of a protein or polypeptide sequence. The nucleic acid construct may comprise DNA and/or RNA (preferably, DNA) and may include regions that are single stranded, double stranded (through base pair binding to its complementary sequence), or partially double stranded. The nucleic acid constructs may contain an RNA polymerase promoter sequence that directs the activity of a RNA polymerase to copy at least a portion of the nucleic acid construct to produce an RNA transcript. This transcript, preferably, either encodes a protein or polypeptide or a portion of a protein or polypeptide. The proteins or polypeptides may correspond to amino acid sequences that are found in nature or may be man-made sequences (i.e., sequences not found in nature). The nucleic acid constructs may include synthetic nucleic acid analogs or derivatives that are capable of being copied by transcriptional machinery.

In one aspect, the present invention contemplates the use of protein tags. A “protein tag” is a biochemical indicator. Protein tags, such as, e.g., affinity tags appended to recombinantly expressed proteins, can serve several purposes, and can be used as a way of purifying proteins using standard conditions rather than developing individual biochemical purifications based on each protein's physical characteristics. For example, a c-Myc-tag can be used for such purposes. The role of tags as an aid to solubilization of a fusion partner has been exploited and maltose binding protein (MBP), glutathione-S-transferase (GST) and thioredoxin have proved useful in this regard. Some tags can be used as an indicator of fusion protein folding—most notably the Green Fluorescent Protein (GFP). Tags are also useful in providing a common epitope, allowing a single antibody to recognize each fusion protein. Some tags are multifunctional, combining two or more of these roles; for example, the his-tag both permits purification on Ni²⁺-NTA and is used as a common epitope, while GST solubilizes some fusion partners, often increases expression levels, permits purification on glutathione-sepharose and provides a common epitope. The Biotin Carboxyl Carrier Protein (BCCP) tag has found particular utility in array fabrication protocols. This tag is a 79-residue polypeptide derived from the biotin carboxyl carrier domain of the E. Coli ACCB protein and is efficiently biotinylated in vivo at a single surface-exposed lysine residue by both prokaryotic and eukaryotic biotin ligases.

The terms “isolated”, “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A peptide or protein that is the predominant species present in a preparation is substantially purified. The term “purified” denotes that a peptide or protein gives rise to essentially one band in an electrophoretic gel or HPLC spectrum. Particularly, it means that the peptide or protein is at least 85% pure, more preferably at least 95% pure, and most preferably at least 99% pure.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

“Substantial identity” of polynucleotide sequences means that a polynucleotide comprises a sequence that has at least 25% sequence identity. Alternatively, percent identity can be any integer from 25% to 100%. More preferred embodiments include at least: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98% or 99% compared to a reference sequence using the programs described herein; preferably BLAST using standard parameters, as described. One of skill will recognize that these values can be appropriately adjusted to determine corresponding identity of proteins encoded by two nucleotide sequences by taking into account codon degeneracy, amino acid similarity, reading frame positioning and the like.

“Substantial identity” of amino acid sequences for purposes of this invention normally means polypeptide sequence identity of at least 40%. Preferred percent identity of polypeptides can be any integer from 40% to 100%. More preferred embodiments include at least 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99%. Polypeptides that are “substantially identical” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine.

A “functional fragment” or “functional equivalent” or “functional homolog” of a polypeptide of the present invention is a polypeptide that is homologous to the specified polypeptide but has one or more amino acid differences from the specified polypeptide. A functional fragment or equivalent of a polypeptide retains at least some, if not all, of the activity of the specified polypeptide.

“Preventing” or “prevention” as used herein refers to the preventing or prevention of a disease or medical condition. Examples of a disease or a medical condition are Alzheimer's disease (also simply known as Alzheimer's or AD) and other neurodegenerative diseases. The compositions and methods of the present invention can be used for prevention of both Alzheimer's and other neurodegenerative diseases. Therefore, as used herein, reference to “prevention of Alzheimer's” is meant to also include the prevention of other neurodegenerative diseases.

“Treating” or “treatment” as used herein refers to the treating or treatment of a disease or medical condition. Examples of a disease or a medical condition are Alzheimer's and other neurodegenerative diseases. Therefore, as used herein, reference to “treatment of Alzheimer's” is meant to also include the treatment of other neurodegenerative diseases. The terms “treating” or “treatment” as used herein include achieving a therapeutic benefit and/or a prophylactic (preventative) benefit.

In one aspect, the present invention provides for the identification of the first known two ER-based acetyltransferases, named ATase1 and ATase2. Both proteins display acetyCoA:lysine acetyltransferase activity in vitro, can interact with and acetylate BACE1 in vitro and in vivo, and show a predominant ER/ERGIC localization. Both ATase1 and ATase2 regulate the steady-state levels of BACE1 and the rate of Aβ generation, and are upregulated in the brain of late-onset AD patients. The acetylCoA:lysine acetyltransferase activity of ATase1 and/or ATase2 can be determined in a variety of ways, e.g., as described in the examples section below, and also as described in Costantini et al., 2007, Biochem. J. 407: 383-395 and in Ko and Puglielli (manuscript submitted).

In some embodiments, the present invention provides targets for the prevention and treatment of Alzheimer's disease. For example, the acetylCoA:lysine acetyltransferase activity of ATase1 is used as a target for the pharmacological prevention and treatment of Alzheimer's disease and related neurodegenerative diseases. Alternatively, the acetylCoA:lysine acetyltransferase activity of ATase1 is used as a target for the pharmacological prevention and treatment of Alzheimer's disease and related neurodegenerative diseases. As well, the acetylCoA:lysine acetyltransferase activity of both ATase1 and ATase2 is used as a target for the pharmacological prevention and treatment of Alzheimer's disease and related neurodegenerative diseases. Drugs (e.g., inhibitors, agonists, promoters, etc.) are thus identified and/or designed, which will target the acetylCoA:lysine acetyltransferase activity of ATase1, the acetylCoA:lysine acetyltransferase activity of ATase2, or the acetylCoA:lysine acetyltransferase activity of both ATase1 and ATase2. In some embodiments, screening assays are designed to identify compounds that decrease or inhibit the acetyltransferase activity of ATase1 and/or ATase2. Such compounds are useful for the prevention and/or treatment of Alzheimer's disease and related neurodegenerative diseases. The acetylCoA:lysine acetyltransferase activity of ATase1 and ATase2 described herein occur downstream of the aging program mediated by IGF1-R, the common regulator of lifespan in all organisms. The regulation of the rate of amyloid β-peptide generation during aging suggests an immediate implication in Alzheimer's disease (AD) and other age-associated diseases.

Using the methods of the present invention, BACE1 stability is altered through modulating levels of acetylCoA:lysine acetyltransferase activity of ATase1, ATase2, or both ATase1 and ATase2. Increasing the levels of acetylCoA:lysine acetyltransferase activity of ATase1, ATase2, or both ATase1 and ATase2, stabilizes BACE1, leading to greater accumulation of beta amyloid, which is associated with Alzheimer's disease. Conversely, decreasing the levels of acetylCoA:lysine acetyltransferase activity of ATase1, ATase2, or both ATase1 and ATase2, destabilizes BACE1, which decreases the accumulation of beta amyloid, which is associated with Alzheimer's disease. Thus, decreasing the levels of acetylCoA:lysine acetyltransferase activity of ATase1, ATase2, or both ATase1 and ATase2, results in the prevention and/or treatment of Alzheimer's disease and related neurodegenerative diseases.

The methods of the present invention may be carried out using cell extracts. Alternatively, the methods of the present invention may be carried out using ER-derived vesicles (ER vesicles) and Golgi-derived vesicles (Golgi vesicles).

The methods of the present invention may be carried out using calorimetric assays, measuring incorporated radioactivity (e.g. with a scintillation counter), etc. For example, the acetyltransferase activity of ATase1 and ATase2 may be determined using calorimetric assays as described below. Such calorimetric assays can be used, for example, to run a high-throughput screen for inhibitory compounds.

In some embodiments, the methods of the present invention may typically employ affinity purified acetylCoA:lysine acetyltransferase (ATase1, ATase2, or both ATase1 and ATase2), in the amount of approximately 10 μl of the 600 μl that comes out of a 20 μl affinity column, as described below; a similar amount of acceptor-substrate (e.g. BACE1) is also used; the amount of donor-substrate is about 5-30 μM; the pH range for the assays is typically between 7.0 and 7.4; the assays are typically carried out at a temperature of 25-30° C. While a variety of acetyl donors may be used for practicing the invention, a preferred acetyl donor is acetyl coenzyme A (acetylCoA, AcCoA, acetylCoA).

In one aspect of the present invention, provided are methods for treating Alzheimer's disease. The methods include reducing the acetylCoA:lysine acetyltransferase activity of a polypeptide that is at least 95% identical to SEQ ID NO:1 or SEQ ID NO:2 in the endoplasmic reticulum of a subject with Alzheimer's disease. The methods may include administering a therapeutically effective amount of an inhibitor of acetylCoA:lysine acetyltransferase activity to the subject, where the inhibitor of acetylCoA:lysine acetyltransferase activity reduces or eliminates the acetylCoA:lysine acetyltransferase activity in the endoplasmic reticulum.

In another aspect, the methods for treating Alzheimer's disease may include reducing the acetylCoA:lysine acetyltransferase activity of an acetylCoA:lysine acetyltransferase from the ER in a subject with Alzheimer's disease. The acetylCoA:lysine acetyltransferase may be ATase1. Alternatively, the acetylCoA:lysine acetyltransferase may be ATase2. The acetylCoA:lysine acetyltransferase may acetylate BACE1.

In one example, decreasing the gene expression of ATase1, ATase2, or both ATase1 and ATase2 is beneficial for the prevention and/or treatment of Alzheimer's disease and related neurodegenerative diseases.

In another example, inhibiting or decreasing the protein expression of ATase1, ATase2, or both ATase1 and ATase2 is beneficial for the prevention and/or treatment of Alzheimer's disease and related neurodegenerative diseases.

In yet another example, inhibiting or decreasing the acetyltransferase activity of ATase1, ATase2, or both ATase1 and ATase2 is beneficial for the prevention and/or treatment of Alzheimer's disease and related neurodegenerative diseases. In particular, decreasing the acetylCoA:lysine acetyltransferase activity of ATase1, ATase2, or both ATase1 and ATase2 is beneficial for the prevention and/or treatment of Alzheimer's disease and related neurodegenerative diseases.

The methods of this invention provide a facile and specific assay to screen compounds as potential Alzheimer's drugs. In the present invention ATase1, ATase2, or both ATase1 and ATase2 may be purified or partially purified native proteins, cloned proteins or engineered variants thereof, crude preparations of the ATase1 and/or ATase2 proteins, or extracts containing ATase1 and/or ATase2 activity. The proteins ATase1 and ATase2 are preferably from a mammalian (e.g., human, non-human animal, etc.) source. Fragments of ATase1 and ATase2 that retain ATase1 and ATase2 activity are also within the scope of the invention.

A compound that interacts with ATase1, ATase2, or both ATase1 and ATase2 may be one that is a substrate for ATase1, ATase2, or both ATase1 and ATase2, one that binds ATase1, ATase2, or both ATase1 and ATase2, or one that otherwise acts to alter ATase1, ATase2, or both ATase1 and ATase2 activity by binding to an active or an alternate site. The compound that interacts with ATase1, ATase2, or both ATase1 and ATase2 can be labeled to allow easy quantitation of the level of interaction between the compound and ATase1, ATase2, or both ATase1 and ATase2. Examples of useful radiolabels are tritium or ¹⁴C.

“Therapeutically effective amount” refers to that amount of a composition resulting in amelioration of symptoms or a prolongation of survival in a patient. A therapeutically relevant effect relieves to some extent one or more symptoms of a disease or condition or returns to normal either partially or completely one or more physiological or biochemical parameters associated with or causative of the disease or condition.

In some embodiments, the present invention provides methods for designing in vitro assays for the presence of a known target in Alzheimer's disease. Examples of such targets are ATase1, ATase2, or both ATase1 and ATase2. The invention provides methods for assaying for the identification of modulators of post-translational modification pathways, and in particular acetylCoA:lysine acetyltransferase activity, that play a role in Alzheimer's progression. These modulators may be novel drugs for the prevention or treatment of Alzheimer's. Alternatively, the discovery of such modulators may lead to the discovery of new drug targets for Alzheimer's. For example, such modulators may include ATase1 ligands, ATase2 ligands, or both ATase1 and ATase2 ligands. The modulators may also be ATase1 inhibitors, ATase2 inhibitors, or both ATase1 and ATase2 inhibitors. Conducted in vitro, such assays may include post-translation modification of proteins using ER-derived vesicles. ER-derived vesicles and Golgi-derived vesicles can be obtained using methods known in the art, e.g. as described in the examples section below, and also as described by Kuehn et al., 1998, Nature 391: 187-190; Costantini et al., 2006, EMBO J. 25: 1997-2006.

In some embodiments, the present invention contemplates screens for compounds that can be used as inhibitors, ligands, or promoters of the acetylCoA:lysine acetyltransferase activity of ATase1, ATase2, or both ATase1 and ATase2, described herein. As well, these compounds may find use for developing drugs or treatments for Alzheimer's disease. In general, compounds are identified from libraries of both natural product extracts (e.g. obtained from plant, microorganism or animal sources) and synthetic (or semi-synthetic) extracts or chemical libraries according to methods known in the art. The precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), and Harbor Branch Oceanographic Institute (Ft. Pierce, Fla.). Natural and synthetically produced libraries are produced, if desired, according to methods known in the art, e.g., by extraction and fractionation. If desired, any library or compound is readily modified using standard chemical, physical, or biochemical methods.

When a crude extract is found to decrease activity of the tested enzyme (e.g., ATase1, ATase2, or both ATase1 and ATase2) in the ER, further fractionation of the positive lead extract is typically necessary to isolate chemical constituents responsible for the observed effect. Thus, the goal of the extraction, fractionation, and purification process is the careful characterization and identification of a chemical entity within the crude extract having the desired activity. Methods of fractionation and purification of such heterogeneous extracts are known in the art. If desired, compounds shown to be useful agents for the treatment of pathogenicity may be further chemically modified according to methods known in the art.

One embodiment of the present method is based on testing compound-induced inhibition of ATase1, ATase2, or both ATase1 and ATase2 activity. The ATase1, ATase2, or both ATase1 and ATase2 inhibition assay involves adding ATase1, ATase2, or both ATase1 and ATase2 or an extract containing ATase1, ATase2, or both ATase1 and ATase2 to mixtures of ATase1, ATase2, or both ATase1 and ATase2 substrate (e.g., BACE1) and the test compound, both of which are present in known concentrations. The assay is carried out with the test compound at a series of different dilution levels. After a period of incubation, the labeled portion of the substrate released by the ATase1, ATase2, or both ATase1 and ATase2 enzymatic action is separated and counted. The assay is generally carried out in parallel with a control (no test compound) and a positive control (containing a known enzyme inhibitor instead of a test compound).

In some aspects of the present invention, provided are methods for assaying for ATase1, ATase2, or both ATase1 and ATase2 inhibitors, ligands, or promoters. The screens can be conducted to identify compounds such as drugs that can block or diminish ATase1, ATase2, or both ATase1 and ATase2 activity. Such ATase1, ATase2, or both ATase1 and ATase2 inhibitors (e.g., ATase1, ATase2, or both ATase1 and ATase2-inhibiting drugs) can help prevent the buildup of beta-amyloid. Because accumulation of beta amyloid is associated with Alzheimer's disease, compounds that block or diminish ATase1, ATase2, or both ATase1 and ATase2 activity and may help slow or stop the Alzheimer's disease.

In certain preferred embodiments of the invention, translation and, optionally, transcription of nucleic acid constructs to produce proteins (e.g. ATase1, ATase2, or both ATase1 and ATase2) is carried out in an assay plate used for carrying out an assay for an activity of interest. Nonlimiting examples include plates having assay domains coated with (i) a substrate of an activity, (ii) a binding reagent specific for a substrate of an activity, (iii) a binding reagent specific for a product of an activity, (iv) a binding reagent specific for a post-translational modification of the translation products, (v) a binding reagent suitable for immobilizing the translation products, etc.). The assays of the invention are then carried out in this same plate. Alternatively, in other preferred embodiments, the translation reaction is carried out in one container, e.g., the well of a multi-well plate and the translation products are transferred to an assay plate for analysis.

The assays of the invention may detect or utilize binding interactions of binding species. “Binding species”, as used herein, is a term used to describe a molecular species that is able to bind to another molecular species, its binding partner. These binding interactions are characterized in that they are non-covalent, or covalent having dissociation constants which are, preferably, lower than 1 mM, more preferably lower than 100 μM, more preferably lower than 10 μM, and most preferably lower than 1 μM. Examples of binding species and binding partners include, but are not limited to, biotin, antibodies, streptavidin, avidin, EDTA, chelators (e.g., EDTA, NTA, IDA, etc.), antigens, fluorescein, haptens (e.g., fluorescein, digoxigenin, etc.), proteins, peptides, drugs, nucleic acids, nucleic acid analogues, lipids, carbohydrates, protein A, protein G, protein L, receptors, ligands, inhibitors, lectins, enzymes, substrates, transition state analogues, mechanism based inhibitors, epitopes, affinity tags (e.g., epitope tags such as his(6), glutathione, Myc, S-tag, T7-Tag), etc.

The assays of the invention may utilize labels. “Label” or “detectable label” as used herein is used to describe a substance used to detect (directly or indirectly) a molecular species. The label may be the molecular species itself or it may be linked to the molecular species. In certain embodiments of the invention, labels are used in order to follow or track a given molecular species, for example, to determine its distribution and concentration (as, for example, a radio-labeled drug molecule is used to determine its pharmacological properties when introduced into an animal or patient). In alternative embodiments, a label is introduced into a binding species so as to allow the binding species to be used to track and/or determine the presence and/or amount of a binding partner of the binding species. For example, immunoassays using labeled antibodies (e.g., antibodies labeled with ECL labels) can be used to detect and determine binding partners (analytes) bound by the antibody. The term “label” also includes indirect labeling of proteins using detectable labels bound to other molecules or complexes of molecules that bind to a protein of interest, including antibodies and proteins to which antisera or monoclonal antibodies specifically bind. As used herein, the term “calorimetric label” includes a label that is detected using an enzyme-linked assay.

In some embodiments of the invention, labels are used which may be detected directly, e.g., on the basis of a physical or chemical property of the label (such as optical absorbance, fluorescence, phosphorescence, chemiluminescence, electrochemiluminescence, refractive index, light scattering, radioactivity, magnetism, catalytic activity, chemical reactivity, etc.). Examples of directly detectable labels include radioactive labels, fluorescent labels, luminescent labels, enzyme labels, chemiluminescent labels, electrochemiluminescent labels, phosphorescent labels, light scattering or adsorbing particles (e.g., metal particles, gold colloids, and silver colloids), magnetic labels, quantum dots, etc. In other embodiments of the invention, labels are used which may be detected indirectly via interactions with species comprising directly detectable labels. The indirectly detectable labels are, preferably, binding species; these binding species readily allow the binding to a binding partner that is labeled with a directly detectable label. Examples of indirectly detectable labels include binding species as described above, e.g., antibodies, antigens, haptens, avidin, biotin, streptavidin, fluorescein, nucleic acid sequences, nucleic acid analogue sequences, epitope tags (such as myc, FLAG, GST, MBP, V5), digoxigenin, etc.

The invention relates, in part, to the production of proteins in cell-free systems and the analysis of these proteins. Protein synthesis can be accomplished using cell lysates or extracts (crude or partially purified) that contain the machinery necessary for protein synthesis. This machinery is found in most living cells; however, certain cell types are preferred because of their high protein synthesis activity. Three preferred translation systems for producing proteins are the (i) bacterial (more preferably E. Coli extract, most preferably E. Coli S30 cell free extract), (ii) plant germ (most preferably, wheat germ), and (iii) reticulocyte (most preferably, rabbit reticulocyte) lysate translation systems. Optionally, the cell lysates are supplemented with additional components such as ATP, tRNA, amino acids, RNA polymerases, microsomes, protease inhibitors, proteosome inhibitors, etc., that enhance the functioning of the translation machinery, provide a missing component of the machinery, inhibit protein degradation, or provide an additional activity such as transcription or post-translational modification. In an alternate embodiment of the invention, the cell-free system is reconstituted from individual purified or partially purified components, e.g., reconstituted E. coli translation system using recombinant components as described in Shimizu et al., 2001, Nat. Biotech. 19: 751-755. In another alternate embodiment, a cell-based translation system is used. The E. coli systems are advantageous when a gene has been cloned into a vector with prokaryotic regulatory sequences, such as the promoter and ribosome binding site. These systems are coupled in that transcription and translation can occur simultaneously. The E. coli extract systems have been the subject of much optimization and allow the production of mg amounts of protein using large scale (1 ml) reactions fed with reagents through semi-permeable membranes.

Wheat germ and rabbit reticulocyte based cell free systems are also suitable for the translation of mRNA into proteins. These lysates can be supplemented with RNA polymerases so that they carry out both transcription, to generate the mRNA, and translation, to produce protein, thus producing what is called a coupled system. The lysates based on the wheat germ and the rabbit reticulocyte may also be supplemented with other components in order to introduce additional activities to these lysates. The lysate may be supplemented with microsomes (preferably dog pancreatic microsome preparations) or Xenopus oocyte extract to allow for certain post-translation modifications as well as the processing of proteins which are secreted, inserted, or associated with membranes.

In some embodiments of the invention, solid phase supports are used for purifying, immobilizing, or for carrying out solid phase activity assays for analyzing the activity of one or more expressed proteins. Examples of solid phases suitable for carrying out the methods of the invention include beads, particles, colloids, single surfaces, tubes, multiwell plates, microtitre plates, slides, membranes, gels and electrodes. When the solid phase is a particulate material it is, preferably, distributed in the wells of multi-well plates to allow for parallel processing of the solid phase supports. Proteins of interest or other assay reagents may be immobilized on the solid phase supports, e.g., by non-specific adsorption, covalent attachment or specific capture using an immobilized capture reagent that binds, preferably specifically, the protein or assay reagent of interest. Immobilization may be accomplished by using proteins or assay reagents that are labeled with binding species that form binding pairs with immobilized capture reagents. Optionally, a protein is immobilized on a solid phase, then the solid phase is washed and the protein is analyzed. The wash step allows for the rapid purification of the protein from other, potentially interfering components of the translation reaction. Optionally, a protein is treated, prior to analysis, to add or remove post-translational modifications.

In an embodiment of the invention, a series of nucleic acid constructs is obtained with the genes of interest located within the construct such that the gene of interest can be transcribed into RNA that can direct the synthesis of the protein(s) encoded by the genes of interest. The nucleic acid from these is then subjected to an in vitro transcription reaction and followed by an in vitro translation reaction. These two reactions can also be combined in a single in vitro reaction. During the translation reaction the protein(s) produced in the transcription and translation reaction can also be labeled using a modified tRNA that directs the incorporation of a modified amino acid during this step.

Following the transcription and translation reactions the proteins produced in the mix may be captured onto a solid phase. Examples of solid phases suitable for carrying out the methods of the invention include beads, particles, colloids, single surfaces, tubes, multiwell plates, microtitre plates, slides, membranes, gels and electrodes. The capture of the proteins is typically achieved using a binding partner specific for the modification introduced by the modified tRNA during the translation reaction. A good example of how this is achieved is by the use of the biotin-lys-tRNA that results in a protein containing lysine residues modified with biotin. The biotinylated proteins produced in this way are then captured onto a solid phase using an avidin or streptavidin coated solid phase. Alternative methods for capture of the proteins produced include non-specific or passive adsorption, and via the binding to specific binding partners other than those for the modified amino acids introduced by the addition of modified tRNA (e.g., binding partners of affinity tags present in the expressed protein). Alternatively, the proteins produced may be generated without the use of the modified tRNA and they may be captured using alternative methods for capture, including non-specific or passive adsorption, and via binding to specific binding partners for the produced proteins (e.g., binding partners of affinity tags present in the expressed proteins).

In certain embodiments of the invention, a protein expressed by the expression methods of the invention is characterized by its catalytic activity or its ability to act as a substrate in the presence of a catalytic activity or other chemical activity. In certain other embodiments of the invention, an activity is measured using a substrate that exhibits a detectable change in a chemical or physical property when acted on by the activity of interest (e.g., luminescence, color, ability to act as an ELC coreactant, mobility shift on a gel, mass spectrometry, etc.).

In one embodiment of the invention, a nucleic acid construct is preferably obtained with the genes of interest located within the construct such that the gene of interest can be transcribed into RNA that can direct the synthesis of the protein encoded by the genes of interest. The nucleic acid construct is then subjected to an in vitro transcription reaction and followed by an in vitro translation reaction. These two reactions can also be combined in a single in vitro transcription/translation reaction. The protein product (or, alternatively, protein obtained by any other method, e.g. purified) is then analyzed for the regulation of acetyltransferase activity of ATase1, ATase2, or both ATase1 and ATase2. Preferably, a plurality of constructs comprising different genes are prepared and the protein products of these genes produced and analyzed, most preferably in parallel in the wells of multi-well plates.

In one example, the assays of acetyltransferase activity of ATase1, ATase2, or both ATase1 and ATase2, are carried out by compounds located in ER vesicles, Golgi vesicles, or combinations of ER vesicles and Golgi vesicles, and this is readily detected using antibodies specific to acetylation. In one example an antibody is labeled with a detectable label and the bound signal detected to determine the amount of acetylation for a given protein. Alternatively, the assays of acetyltransferase activity of ATase1, ATase2, or both ATase1 and ATase2 of the present invention can be detected by mass spectrometry using methods known in the art.

In a further embodiment of the invention, a labeled substrate for acetylation is included in the translation reaction. An example of such substrates includes labeled BACE1. Examples of labels that could be used include, but are not limited to, metal chelators, biotin, binding species, digoxigenin, enzymes, fluorophores, luminescent species, and ECL labels. The result of the inclusion of a labeled substrate for acetylation reaction is that those proteins that are subjected to this acetylation are produced with the acetylation including the label. This results in the production of a labeled protein. Proteins that are labeled in this way through the acetylation reaction or are labeled as described using modified tRNA contain two, preferably different, labels. These proteins are readily detected by immobilization through one of the labels and detection with the other.

In an alternative embodiment, the invention involves either obtaining clones or the production of clones in a desired cloning vector or nucleic acid construct. The DNA from these is then subjected to an in vitro transcription reaction and followed by an in vitro translation reaction. These two reactions can also be combined in a single in vitro transcription/translation reaction. During the translation reaction the protein produced (e.g., ATase1, ATase2) is also labeled using a modified tRNA that directs the incorporation of a modified amino acid during this step. On completion of this protein expression step the reaction mix is subjected to assays for acetyltransferase activity. The protein in the mix is then captured onto a solid phase using a binding protein specific for the acetylation. A good example of how this is achieved is by the use of an antibody specific for acetylation, for example an anti-acetylated Lysine antibody that is immobilized onto a solid phase. This allows the capture of proteins that have been acetylated onto the solid phase. Examples of the solid phase include beads, particles, colloids, single surfaces, tubes, multiwell plates, microtitre plates, slides, membranes, gels and electrodes. With these proteins immobilized onto a solid phase the proteins are then available for an assay to determine if they have been captured and thus subject to any structural modifications or acetylation. These proteins are then detected using a binding species specific for the modified amino acid that has an attached label.

The nucleotide and amino acid sequences of ATase1 can be found in GenBank under accession number NM_{—}016347. The amino acid sequence of the ATase1 polypeptide is shown as SEQ ID NO:1.

The nucleotide and amino acid sequences of ATase2 can be found in GenBank under accession number NM_{—}003960. The amino acid sequence of the ATase2 polypeptide is shown as SEQ ID NO:2.

The coding sequence of ATase1 is shown as SEQ ID NO:3. The coding sequence of ATase2 is shown as SEQ ID NO:4.

As used herein, the phrase “nucleic acid”, “nucleotide sequence” or “polynucleotide sequence” refers to a single or double-stranded polymer of deoxyribonucleotide or ribonucleotide bases read from the 5′ to the 3′ end. Nucleic acids may also include modified nucleotides that permit correct read-through by a polymerase and do not alter expression of a polypeptide encoded by that nucleic acid. A “coding sequence” or “coding region” refers to a nucleic acid molecule having sequence information necessary to produce a gene product, when the sequence is expressed. The phrase “nucleic acid sequence encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the full length nucleic acid sequences as well as non-full length sequences derived from the full length sequences. It should be understood that the sequence includes the degenerate codons of the native sequence or sequences which may be introduced to provide codon preference in a specific host cell.

The polypeptide sequences of ATase1 and ATase2 have about 87-88% identity while exhibiting identical enzymatic activities. ATase1 and ATase2 are substantially identical, as described above. Because the polypeptide sequences of ATase1 and ATase2 are 87% homologous while maintaining identical enzymatic activities, the present invention contemplates the use of polypeptides with variable amino acid sequences. Therefore, polypeptides useful for practicing the present invention may have substantial identity to ATase1, i.e. they may have polypeptide sequence identity of at least 87%, 88%, 89%, 90%, 91%, 92%, 93%, 94%, 95%, 96%, 97%, 98%, or 99% relative to ATase1. As well, polypeptides useful for practicing the present invention may have substantial identity to ATase2, i.e. they may have polypeptide sequence identity of at least 87%, 88%, 89%, 90%, 91%, 92%, 93%, 94%, 95%, 96%, 97%, 98%, or 99% relative to ATase2.

### EXAMPLES

It is to be understood that this invention is not limited to the particular methodology, protocols, subjects, or reagents described, and as such may vary. It is also to be understood that the terminology used herein is for the purpose of describing particular embodiments only, and is not intended to limit the scope of the present invention, which is limited only by the claims.

The following examples are offered to illustrate, but not to limit the claimed invention.

Cell Cultures and Cell Treatment

Chinese Hamster Ovary (CHO) cells were grown in DMEM (Gibco BRL) supplemented with 10% fetal bovine serum (FBS) and penicillin/streptomycin (Mediatech, Inc., Herndon, Va.) as described before (Puglielli et al., 2001, Nat. Cell Biol. 3: 905-912; Puglielli et al., 2003, J. Biol. Chem. 278: 19777-19783). Cells were maintained in a humidified atmosphere with 5% CO₂. For ceramide treatment, C6-ceramide (simply called “ceramide”) was administered in the absence of serum and at the final concentration of 10 μM; treatment was started when cells were ˜80% confluent and carried out for the time specified.

### Antibodies and Western Blot Analysis

Western blotting was performed on a 4-12% Bis-Tris SDS-PAGE system (NuPAGE; Invitrogen, Carlsbad, Calif.) as described (Puglielli et al., 2001, Nat. Cell Biol. 3: 905-912; Puglielli et al., 2003, J. Biol. Chem. 278: 19777-19783; Costantini et al., 2007, Biochem. J. 407: 383-395).

The following antibodies were used: anti-BACE1 N-terminal (monoclonal; R&D Systems, Minneapolis, Minn.); anti-BACE1 C-terminal (polyclonal; Abcam, Cambridge, Mass.); anti-acetylated lysine (monoclonal; Abcam); anti-calreticulin (ER marker; polyclonal; Abcam); anti-syntaxin (Golgi marker; monoclonal; Abcam); anti-ERG IC-53 (ERG IC marker; polyclonal; Sigma, St. Louis, Mo.); anti-EEA1 (endosomal marker; monoclonal; BD Transduction Laboratories); anti-myc (polyclonal; Sigma); anti-actin (polyclonal; Cell Signaling, Danvers, Mass.).

Secondary antibodies (Amersham) were used at a 1:6000 dilution. Binding was detected by chemiluminescence (LumiGLO kit; KPL, Gaithersburg, Md.).

Cell Extraction, Immunoprecipitation, and Affinity Purification

Cell lysates were prepared in GTIP buffer (100 mM Tris-pH 7.6, 20 mM EDTA, 1.5 M NaCl) with 1% Triton X-100 (Roche, Basel, Switzerland), 0.25% NP40 (Roche), and a protease inhibitor cocktail (Roche) as described before (Puglielli et al., 2001, Nat. Cell. Biol. 3: 905-912; Puglielli et al., 2003, J. Biol. Chem. 278: 19777-19783; Costantini et al., 2007, Biochem. J. 407: 383-395). Briefly, cell monolayers were washed twice in PBS, scraped, transferred into eppendorf tubes, and centrifuged at 8,000 rpm for 5 min in a table-top centrifuge. Cell pellets were then lysed in ice for 30 min with the buffer indicated above, and centrifuged again at 14,000 rpm prior to the recollection of the protein extracts. Proteins were quantitated with the BCA Protein Assay (Pierce, Rockford, Ill.) following the manufacturer's instructions.

For the immunoprecipitation of BACE1, cell lysates were pre-cleared with BioMag Protein A (Polysciences, Inc., Warrington, Pa.); beads were used at the concentration of 15 μl of the slurry per sample and separated with a magnetic holder. BACE1 was then immunoprecipitated with anti-BACE1 antibodies overnight and recovered with BioMag Protein A as above. The immunoprecipitate-bead complexes were washed 3 times in PBS and boiled for 10 min in SDS sample buffer (Invitrogen) with β-mercaptoethanol. Beads were then separated and the samples were analyzed by reducing SDS-PAGE.

For the affinity purification of BACE1-myc and ATases-myc, the ProFound c-Myc-Tag IP/Co-IP Kit (Pierce) was used as suggested by the manufacturer and previously described (Costantini et al., 2007, Biochem. J. 407: 383-395). Briefly, cell lysates from stably-transfected CHO cells were loaded onto an anti-myc immobilized column (ProFound system), washed, eluted by lowering the pH to 2.0, and recovered by centrifuging at 3000 rpm for 2 min. The pH was immediately neutralized by adding 10 μl of neutralizing buffer (1 M Tris, pH 9.5) per 200 μl of elution buffer.

AcetylCoA:Lysine Acetyltransferase Activity

For the analysis of the acetyltransferase activity, affinity purified BACE1 was employed as acceptor of the acetyl group and [³H]acetylCoA (1000 cpm/pmol) as donor of the acetyl group. The acetylCoA:lysine acetyltransferases (ATase1 and/or ATase2) were provided either as pure enzymes following affinity purification with the ProFound Kit (described above) or as membrane extracts of stably-transfected CHO cells.

The reaction was performed in 150-200 μl of acetylation buffer, consisting of 50 mM Tris HCl (pH 8.0), 0.1 mM EDTA, 1 mM DTT, 10% glycerol, 20 μM acetylCoA, and let run for 1 hr at 30° C. The reaction was stopped by adding an equal volume of ice-cold buffer and immediate immersion in ice; BACE1 was immunoprecipitated with an anti BACE1 N-terminal monoclonal antibody and then counted on a liquid scintillation counter. The experimental controls are described in the appropriate figure legends.

Subcellular Fractionation

Subcellular fractionation of cultured CHO cells was performed using a protocol that has been previously described (Costantini et al., 2006, EMBO J. 25: 1997-2006; Costantini et al., 2007, Biochem. J. 407: 383-395) with minor modifications. Briefly, confluent cells were washed twice each with ice-cold phosphate-buffered saline and homogenization buffer (10 mM triethanolamine, 10 mM acetic acid, 250 mM sucrose, 1 mM EDTA, and 1 mM dithiothreitol; pH 7.4) plus a protease inhibitor cocktail (Roche) and homogenized using a 25-gauge needle and a tight-pestle Dounce homogenizer. A post-nuclear supernatant resulting from low speed centrifugation (1,000×g for 15 min at 4° C.) was separated by differential centrifugation at 14,000×g, and 100,000×g to yield membrane fractions P2 and P3, respectively. Pooled membrane fractions were layered on top of a step gradient consisting of 24, 19.33, 14.66, and 10% isotonic Nycodenz solutions (made in 0.75% NaCl, 10 mM Tris-pH 7.4, 3 mM KCl, and 1 mM EDTA) and centrifuged at 100,000×g in a SW41 rotor for ˜18 h at 4° C. Fractions were collected from the top to the bottom of the gradient. An average of 50 confluent 150 mm Petri dishes was processed for each gradient. In order to assess the quality of the gradients the latency of ER and Golgi vesicles was analyzed using the glucose-6-phosphatase (Clairmont et al., 1992, J. Biol. Chem. 267: 3983-3990) and the sialyl-transferase (Puglielli and Hirschberg, 1999, J. Biol. Chem. 274: 35596-35600) methods, respectively. The gradients yielded vesicles that were >97% sealed and of the same membrane topographical orientation as in vivo.

Aβ Determination

For Aβ determinations in the conditioned media, CHO cells were plated in 6-well Petri dishes. When 80-90% confluent, cells were washed in PBS and incubated in 1 ml of fresh medium for 48 hr. Secreted Aβ was determined by standard sandwich ELISA as described before (Puglielli et al., 2003, J. Biol. Chem. 278: 19777-19783; Costantini et al., 2005, Biochem. J. 391: 59-67; Costantini et al., 2006, EMBO J. 25: 1997-2006). Antibodies 9131 (for Aβ 1-40) and 9134 (for Aβ 1-42), were used as capture antibodies, and 9154 and 4G8 as biotinylated reporter antibodies. The above antibodies were all from Signet Laboratories, Dedham, Mass. For each sample, the levels of Aβ₄₀, Aβ₄₂, and Aβₜₒₜₐₗ were quantified as triplicate based upon standard curves run (on every ELISA plate) and then expressed as pmol Aβ/mg of protein. Aβ₄₂ was constantly found to be ˜20 to 30% of total Aβ values.

Real-Time PCR

Total RNA from H4 (human neuroglioma) and SH-SY5Y (human neuroblastoma) cells, and from human brains (AD patients and age-matched controls) was extracted and purified using the RNAEasy plus kit (Qiagen, Valencia, Calif.). cDNA was synthesized using the SuperScript double stranded cDNA Synthesis System (Invitrogen) and then PCR-amplified. Primers used for RT-PCR were: GAPDH, Forward primer 5′-GAAGGTGAAGGTCGGAGTC-3′ (SEQ ID NO:5), Reverse primer 5′-GAAGATGGTGATGGGATTTC-3′ (SEQ ID NO:6); ATase1, Forward primer 5′-CGATTACTGAAGCTGCCTCGA-3′ (SEQ ID NO:7), Reverse primer 5′-GGTTTTTTGGCAAGGAACCAC-3′ (SEQ ID NO:8); ATase2, Forward primer 5′-TCCTTGCCAAAAAACCCTGG-3′ (SEQ ID NO:9), Reverse primer 5′-ATGCCCACCACCTTCTCTTCA-3′ (SEQ ID NO:10).

RNA quantification was performed by real-time PCR in an ABI PRISM® 7000 Sequence Detection System using SYBR® Green PCR Master Mix (Applied Biosystems). Amplifications were generated at 2 min at 52° C. and 3 min at 95° C., followed by 40 cycles of denaturations at 95° C. for 15 s, annealing, and synthesis (30 s at 61° C. and 30 s at 72° C.). Delta-delta Ct values were normalized with those obtained from the amplification of GAPDH and were expressed as fold-change over control. The assay was repeated three times, with each assay containing triplicate reactions, and with each assay including an independent amplification of the probes.

Statistical Analysis

Results were typically expressed as mean±S.D. of the indicated number of determinations. The data were analyzed by ANOVA and Student's t-test comparison, using GraphPad InStat3 software available from GraphPad Software, Inc., San Diego, Calif. Statistical significance was reached at p<0.05.

Identification of ATase1 and ATase2 as Two ER/ERGIC AcetylCoA:Lysine Acetyltransferases

The inventor previously demonstrated the presence of an acetylCoA:lysine acetyltransferase activity in the ER/ERGIC system (Costantini et al., 2005, Biochem. J. 391: 59-67). The catalytic site of the putative ER/ERGIC-based acetyltransferase(s) faced the lumen of the organelle. In order to identify the above enzymatic activity, the human genome was searched for proteins displaying structural similarities to the histone acetyltransferase catalytic domain. The search yielded 46 possible candidates, which were then individually screened for the presence of a possible signal peptide and a predicted ER/ERGIC localization. This approach identified two proteins with unknown biochemical function that fulfilled both criteria. The candidates, initially named Camello 2 (accession number NP057431) and Camello 1 (accession number NP003951) have homologs in Xenopus laevis (Popsueva et al., 2001, Dev. Biol. 234: 483-496) but not in C. elegans or D. melanogaster. The biochemical activity of both proteins was explored, and based on the results reported here they were re-named ATase1 and ATase2 to indicate their biochemical function rather than the domain organization.

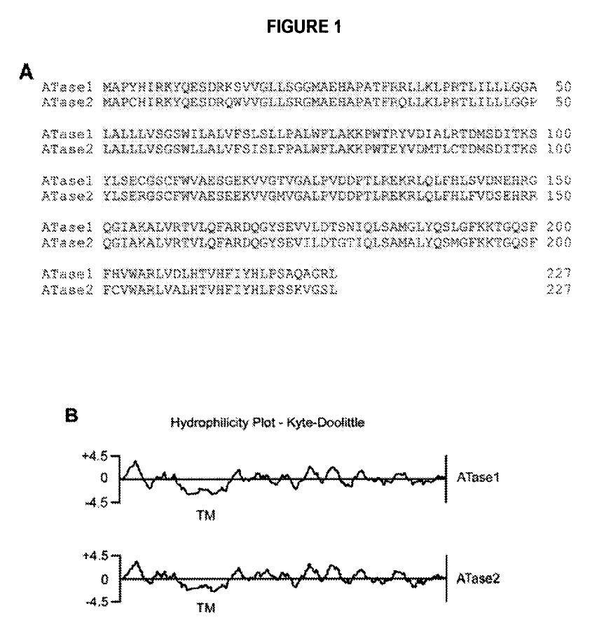

FIG. 1 shows a schematic view of ATase1 and ATase2. FIG. 1 (A) is an alignment of amino acid sequences of ATase1 and ATase2. Alignment was performed using the Megalign component of the DNASTAR (Lasergene-v6) software on a basis of Clustal W algorithm. Identical amino acids are highlighted in yellow. FIG. 1(B) is a hydrophilicity profile (Kyte-Doolittle algorithm) of ATase1 and ATase2, which was calculated using the protean component of the DNASTAR (Lasergene-v6) software. TM indicates the transmembrane (hydrophobic) domain.

Both ATase1 and ATase2 have a predicted signal peptide at the N-terminus (identified with P-NN and P-HMM models using the SignalP 3.0 Server of the Center for Biological Sequence Analysis of the University of Denmark (Emanuelsson et al., 2007, Nat. Protoc. 2: 953-971), which would target them to the secretory pathway. Both ATase1 and ATase2 have a high probability of being ER/ERGIC-resident proteins (k-NN prediction using the PSORT II software of the University of Tokyo, Japan) (Horton et al., 2007, Nucleic Acids Res. 35: W585-587).

Alignment of the amino acid sequences of ATase1 and ATase2 reveals an 87-88% sequence identity between the two proteins (FIG. 1A); the Kyte-Doolittle hydrophilicity/hydrophobicity plot indicates the presence of one single hydrophobic segment, corresponding to the membrane-spanning domain. In both cases the putative catalytic site is situated in the large C-terminal domain and it is predicted to face the lumen of the ER/ERGIC.

ATase1 is an ER/ERGIC Resident AcetylCoA:Lysine Acetyltransferase

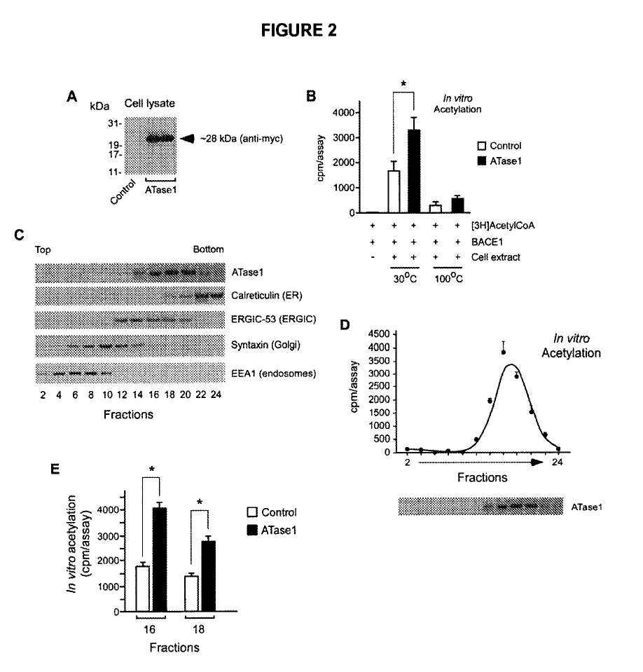

ATase1 was cloned and inserted into a vector for mammalian expression containing a myc tag at the C-terminus to allow subcellular localization and affinity purification for the in vitro studies. FIG. 2 shows how ATase1 is localized in the early secretory pathway and has acetylCoA:lysine acetyltransferase activity. Stable transfection yielded several colonies overexpressing ATase1. Cell extracts from control and ATase1 expressing cells were analyzed in vitro for acetylCoA:lysine acetyltransferase activity. The assay was performed in the presence of affinity purified BACE1, which served as acceptor of the acetyl group, and radiolabeled acetylCoA, which served as donor of the acetyl group. FIG. 2(A) illustrates Western blot analysis of control and ATase1 expressing CHO cells. ATase1 migrates with the expected mass on SDS-PAGE.

FIG. 2B shows that the expression of ATase1 increased the acetyltransferase activity recovered from cell extracts by ˜2-fold. No activity was observed when the assay was performed in the absence of the cell extracts (source of the enzymatic activity) or in the presence of extracts that had been boiled prior to the incubation. In FIG. 2(B), total cell extract from control and ATase1-expressing cells was incubated with [³H]AcetylCoA and affinity purified BACE1 for 1 hour at 30° C. Reaction was stopped by lowering the temperature; BACE1 was then purified again and analyzed on a scintillation liquid counter. As control, BACE1 was also incubated with [³H]AcetylCoA in the absence of cell extract and with a cell extract that had been boiled for 10 min prior to the reaction. Results are the average (n=6)±S.D. Asterisk (*) indicates statistical significance (p<0.005).

Subcellular fractionation studies indicated a predominant ERGIC localization that extended to ER—but not to Golgi—fractions, which is consistent with the previous localization of the ER/ERGIC-based acetylCoA:lysine acetyltransferase activity (Costantini et al., 2007, Biochem. J. 407: 383-395). The subcellular localization of ATase1 overlaps with the compartments where the acetylation of nascent BACE1 occurs and the non-acetylated mutants of BACE1 are retained. Lys-to-Ala (BACE1_{Ala}) and the Lys-to-Arg (BACE1_{Arg}) mutant forms of BACE1 cannot be acetylated in vivo and in vitro, are unable to achieve conformational maturation/stability, are retained in the ER/ERGIC system, and rapidly degraded by a proteasome-independent system (Costantini et al., 2007, Biochem, J. 407: 383-395). FIG. 2(C) shows the subcellular distribution of ATase1, which was analyzed by SDS-PAGE and immunoblotting after separation of intracellular membranes on a 10-24% discontinuous Nycodenz gradient. The appropriate subcellular markers are indicated: calreticulin (ER), ERGIC-53 (ERGIC), syntaxin (Golgi apparatus), EEA1 (endosomes).

The acetylCoA:lysine acetyltransferase activity of the individual fractions from the gradient shown in FIG. 2C was analyzed, and it was found that the distribution pattern of ATase1 completely overlapped with the acetylCoA:lysine acetyltransferase activity (FIG. 2D). FIG. 2(D) illustrates how the fractions showed in (C) were assayed for acetylCoA:lysine acetyltransferase activity as described in (B). [³H]AcetylCoA served as donor of the acetyl group whereas affinity purified BACE1 served as acceptor substrate. Results are the average (n=3)±S.D. The migration pattern of ATase1 shown in (C) is shown here again to allow comparison with the biochemical activity of the single fractions.

When compared to control cells, ERGIC fractions from ATase1-expressing cells displayed increased (˜2-fold) acetyltransferase activity (FIG. 2E). FIG. 2(E) illustrates how fractions 16 and 18 of a subcellular fractionation gradient (see C) from control and ATase1-expressing cells were compared for acetylCoA:lysine acetyltransferase activity. The assay was run as in (D). Results are the average (n=3)±S.D. Asterisk (*) indicates statistical significance (p<0.005).

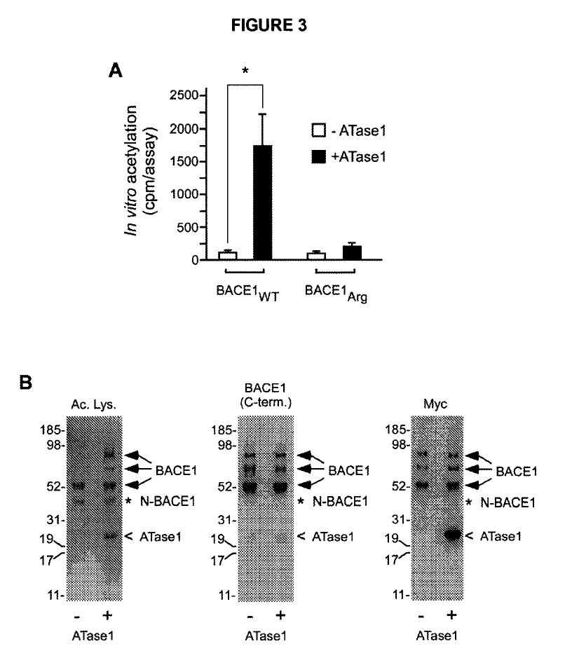

The above results indicate that ATase1 is localized in the compartments where the acetylation of nascent BACE1 normally occurs. They also indicate that the overexpression of ATase1 results in increased acetylCoA:lysine acetyltransferase activity. To rule out the possibility that ATase1 might only be a co-factor in the reaction of lysine acetylation, affinity purified ATase1—instead of membrane extracts—was used for the in vitro acetylation of BACE1. As control, BACE1_{Arg} was used, which has been shown not to act as acceptor of the acetyl groups in vitro or in vivo (Costantini et al., 2007, Biochem. J. 407: 383-395). FIG. 3A shows that purified ATase1 was able to acetylate BACE1_{WT} but not BACE1_{Arg} proving that it is an acetylCoA:lysine acetyltransferase. The results obtained with BACE1_{Arg} are particularly important because they indicate that ATase1 can only acetylate the lysine residues that are found acetylated under normal conditions.

Next, ATase1 was incubated with BACE1 in the presence of non-radiolabeled acetylCoA. Both BACE1 and ATase1 were purified by affinity chromatography prior to the incubation. BACE1 was then immunoprecipitated at the end of the reaction and analyzed by classical immunoblotting.

FIG. 3 illustrates how ATase1 acetylates BACE1 in vitro. FIG. 3(A) shows how affinity purified ATase1 and BACE1 were co-incubated in the presence of [³H]AcetylCoA for 1 hour at 30° C. Reaction was stopped by lowering the temperature; BACE1 was then purified again and analyzed on a scintillation liquid counter. The same assay was repeated with BACE1_{Arg}, a mutant form of BACE1 where the lysine residues that serve as acceptors of the acetyl groups have been mutated to arginine (Costantini et al., 2007, Biochem. J. 407: 383-395). Results are the average (n=6)±S.D. Asterisk (*) indicates statistical significance (p<0.005).

In the absence of ATase1 only the immature form of BACE1 could be detected with an anti-acetylated antibody (FIG. 3B; left panel; —ATase1). FIG. 3(B) shows how affinity purified ATase1 and BACE1 were co-incubated in the presence of acetylCoA (50 μM) for 6 hours at 30° C. Reaction was stopped by lowering the temperature. BACE1 was then immunoprecipitated with a bio-bead cross-linked antibody specific for the N-terminal domain of BACE1 and analyzed by Western blotting with the indicated antibodies (Myc, anti-myc; Ac. Lys., anti-acetylated lysine; BACE1 C-term., anti-BACE1 C-terminal domain). Arrows indicate the different species of BACE1 (immature, partially-glycosylated, and fully-glycosylated). Asterisk (*) indicates a band that could be recognized with an antibody against the N-terminal domain of BACE1 and migrated with the expected molecular mass of the ectodomain of BACE1. Symbol (<) indicates ATase1.

Incubation with ATase1 also allowed detection of two additional bands corresponding to the partially-mature and fully-mature forms of BACE1 (FIG. 3B; left panel; +ATase1). In addition, the intensity of the band corresponding to the immature form increased (FIG. 3B; left panel; +ATase1). These results confirm those performed with radiolabeled acetylCoA (FIG. 3A) and clearly indicate that ATase1 can acetylate BACE1. The normal migration of the immature, partially mature, and mature forms of BACE1 is shown in the central panel of FIG. 3B. The anti-acetylated lysine antibody also detected a lower band migrating with the predicted molecular mass of the ectodomain of BACE1 (FIG. 3B, left panel; indicated by *). The same band could be detected with an antibody against the N-terminal domain of BACE1 (data not shown) but not by antibodies raised against the myc-tag or the C-terminal domain of BACE1 (FIG. 3B, left panel; indicated by *). The band was never observed immediately after purification of BACE1 and appeared only after the 6 hr incubation required for the in vitro acetylation assay, suggesting that it corresponds to the N-terminal ectodomain of BACE1 and most likely results from in vitro autocatalytic cleavage, as previously described (Benjannet et al., 2001, J. Biol. Chem. 276: 10879-10887).

Surprisingly, the anti-acetylated lysine antibody recognized a band of ˜28-kDa, which corresponds to ATase1 (FIG. 3B; left panel; +ATase1). In fact, the same band could be observed with anti-myc antibodies (FIG. 3B; left panel; +ATase1) but not with antibodies against the N-terminal (data not shown) or C-terminal domain (FIG. 3B; middle panel) of BACE1. Therefore, these results indicate that ATase1 can interact with BACE1 in vitro and can undergo acetylation in one or more lysine residues. Not wanting to be bound by the following explanation, a likely explanation is that the acetylation occurs through an autocatalytic mechanism.

ATase2 is an ER/ERGIC Resident AcetylCoA:Lysine Acetyltransferase

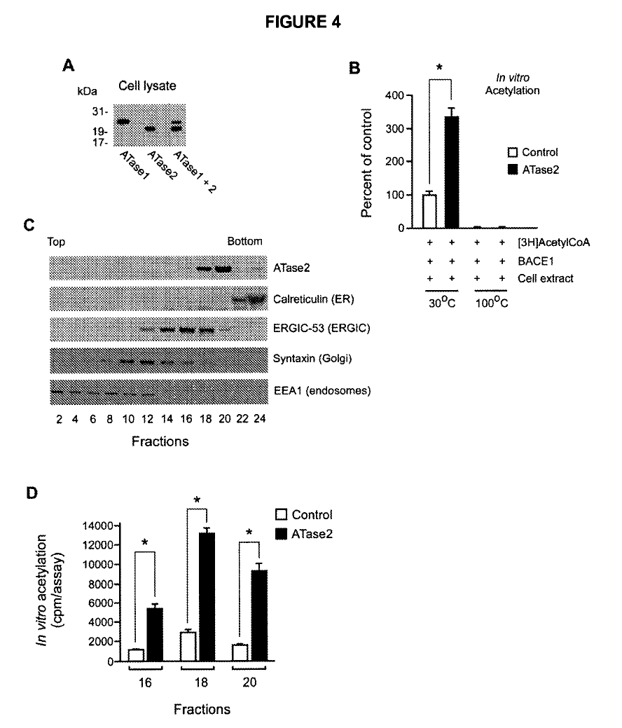

FIG. 4 shows how ATase2 is localized in the early secretory pathway and has acetylCoA:lysine acetyltransferase activity. FIG. 4(A) illustrates Western blot analysis of ATase1-, ATase2-, and both ATase1 and ATase2-expressing CHO cells. In FIG. 4(B), total cell extract from control and ATase2-expressing cells was incubated with [³H]AcetylCoA and affinity purified BACE1 as in FIG. 2B. Reaction was stopped by lowering the temperature; BACE1 was then purified again and analyzed on a scintillation liquid counter. As control, BACE1 was also incubated with a cell extract that had been boiled for 10 min prior to the reaction. Results are expressed as percent of control and are the average (n=6)±S.D. Asterisk (*) indicates statistical significance (p<0.005). In FIG. 4(C), the subcellular distribution of ATase2 was analyzed by SDS-PAGE and immunoblotting after separation of intracellular membranes on a 10-24% discontinuous Nycodenz gradient. The appropriate subcellular markers are indicated: calreticulin (ER), ERGIC-53 (ERGIC), syntaxin (Golgi apparatus), EEA1 (endosomes). In FIG. 4(D), fractions 16, 18, and 20 of a subcellular fractionation gradient (see C) from control and ATase2-expressing cells were compared for acetylCoA:lysine acetyltransferase activity. The assay was performed as in FIG. 2E. Results are the average (n=6)±S.D. Asterisk (*) indicates statistical significance (p<0.005).

ATase2 was cloned and inserted into a vector for mammalian expression containing a myc tag at the C-terminus. This construct was used to generate cells stably expressing ATase2 and ATase1+ATase2 (FIG. 4A). Similarly to ATase1, the expression of ATase2 alone increased (by ˜3-fold) the acetylCoA:lysine acetyltransferase activity recovered from total cell extracts (FIG. 4B). In addition, the subcellular distribution of ATase2 (FIG. 4C)—even though slightly different—appeared to overlap with ATase1 (FIG. 2C) and with the normal distribution of the acetylCoA:lysine acetyltransferase activity (FIG. 2D). Finally, ER/ERGIC fractions generated from ATase2-expressing cells displayed increased acetyltransferase activity, when compared to corresponding fractions from control cells (FIG. 4D).

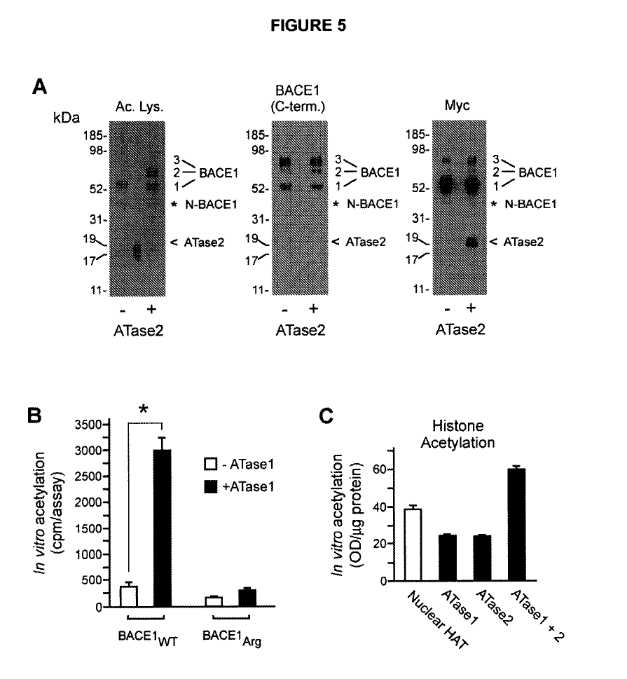

FIG. 5 illustrates how ATase2 acetylates BACE1 in vitro. In FIG. 5(A), affinity purified ATase2 and BACE1 were co-incubated in the presence of acetylCoA (50 μM) for 6 hours at 30° C. This experiment was performed as in FIG. 3B. Following immunoprecipitation with a bio-bead cross-linked antibody specific for the N-terminal domain, BACE1 was analyzed by SDS-PAGE and Western blotting. The following antibodies were used: Myc, anti-myc; Ac. Lys., anti-acetylated lysine; BACE1 C-term., anti-BACE1 C-terminus. Arrows indicate the different species of BACE1 (immature, partially-glycosylated, and fully-glycosylated). Asterisk (*) indicates a band that could be recognized with an antibody against the N-terminal domain of BACE1 and migrated with the expected molecular mass of the ectodomain of BACE1. Symbol (<) indicates ATase2.

In FIG. 5(B), ATase2 was incubated with BACE1 in the presence of [3H]AcetylCoA for 1 hour at 30° C. as in FIG. 3A. Both the enzyme (ATase2) and the acceptor substrate (BACE1) were purified by affinity chromatography prior to the incubation. BACE1_{Arg} was used as control because the lysine residues that serve as acceptors of the acetyl groups have been mutated to arginine. Results are the average (n=6)±S.D. Asterisk (*) indicates statistical significance (p<0.005).

In FIG. 5(C), ATase1 and ATase2 were incubated with BACE1 in the presence of [³H]AcetylCoA for 1 hour at 30° C. as in FIG. 3A. Both the enzyme (ATase1 and ATase2) and the acceptor substrate (BACE1) were purified by affinity chromatography prior to the incubation. BACE1_{Arg} was used as control because the lysine residues that serve as acceptors of the acetyl groups have been mutated to arginine (Costantini et al., 2007, Biochem. J. 407: 383-395). Results are expressed as percent of ATase1 and are the average (n=6)±S.D. Asterisks (*) indicate statistical significance (p<0.005). The average cpm/assay for ATase1 was 1737±486. In FIG. 5(C), the acetylCoA:lysine acetyltransferase activity of ATase1 and ATase2 was assayed with a colorimetric assay employing a pool of histone-tail peptides as acceptor of the acetyl group. Both ATase1 and ATase2 were purified by affinity chromatography prior to the reaction. The acetylCoA:lysine acetyltransferase activity of a highly purified pool of nuclear HAT is also shown. Results are the average (n=3)±S.D.

FIG. 5C shows that both ATase1 and ATase2 displayed robust acetylCoA:lysine acetyltransferase activity, which was in the same range of that observed with HAT. Finally, when ATase1 and ATase2 were included together in the assay, there was a clear additive effect indicating that the two enzymes can act independently of each other.

When affinity purified ATase2 was incubated with BACE1 and non-radiolabeled acetylCoA, there was a marked increase in the acetylation of both the immature and partially-mature forms of BACE1 (FIG. 5A; left panel). Interestingly, the acetylation pattern produced by ATase2 seems somewhat different from the one observed with ATase1 (compare FIG. 5A to FIG. 3B) suggesting potential differences in substrate recognition activities of the two enzymes. Similarly to ATase1, the immunoprecipitation of BACE1 resulted in the pull-down of ATase2, suggesting physical interaction—at least in vitro (FIG. 5A; right panel). However, in contrast to ATase1, it was not possible to clearly resolve a band corresponding to ATase2 with an anti-acetylated lysine antibody (FIG. 5A; left panel). This occurred in the face of the fact that ATase2 could clearly be identified with an anti-myc antibody (FIG. 5A; right panel). Not wanting to be bound by the following theory, these results might indicate potential differences in the biochemical properties and functional regulation of ATase1 and ATase2.

In other experiments, a commercially available colorimetric assay that employs a recombinant peptide corresponding to the N-terminal tail of the histone protein, as acceptor of the acetyl group, and a highly concentrated nuclear histone acetyltransferase (HAT) preparation, as enzyme, were also used. The assay was partially modified in order to employ affinity purified ATases—instead of HAT—as source of the enzymatic activity.

ATase1 and ATase2 Influence the Steady-State Levels of BACE1 and the Generation of Aβ

The results shown in FIGS. 3B and 5A indicate that both ATase1 and ATase2 can be co-immunoprecipitated with BACE1 suggesting physical interaction. However, this experiment was performed after co-incubation of affinity purified ATases and BACE1 in vitro. To assess the physical interaction between the enzyme (ATases) and the substrate (BACE1) in vivo, cell lines that had been transfected with ATase1-myc or ATase2-myc, but not with BACE1-myc, were used, to analyze the possible interaction with endogenous BACE1.

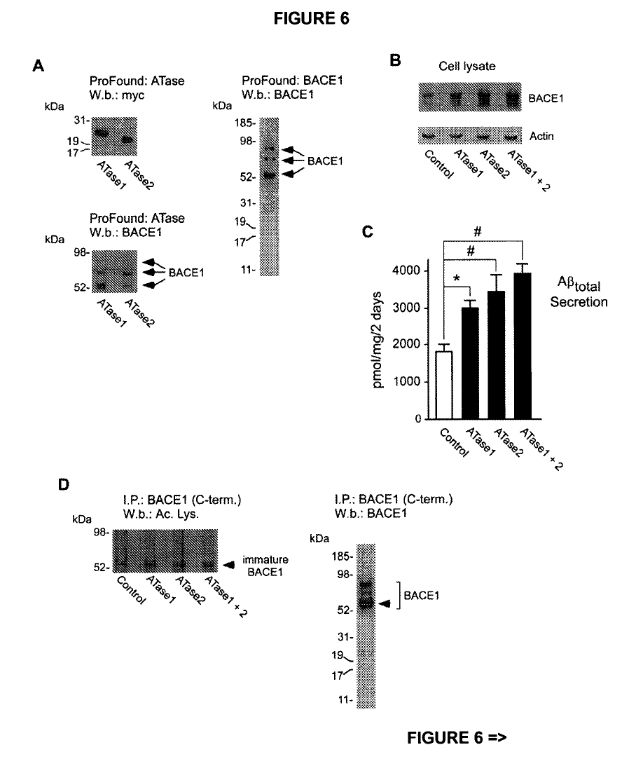

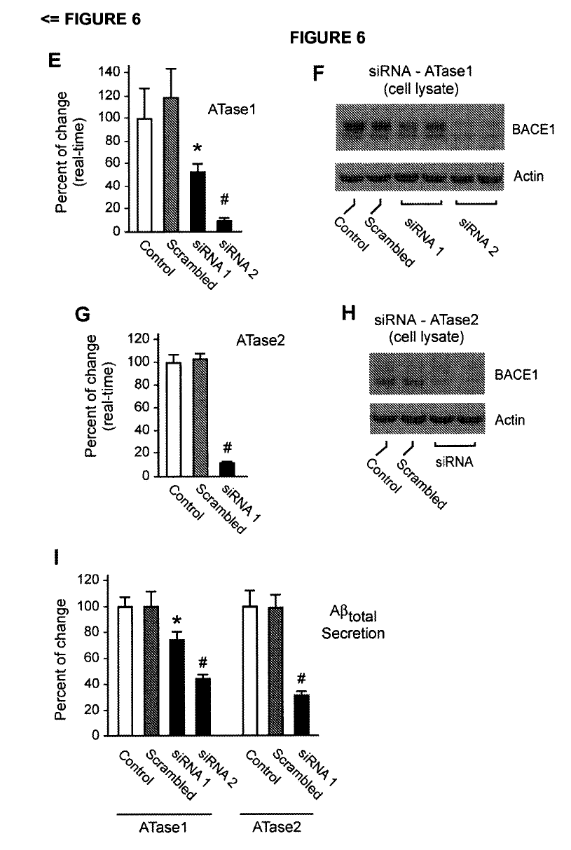

FIG. 6 shows how ATase1 and ATase2 regulate the steady-state levels of BACE1 and the generation of Aβ. In FIG. 6(A), ATase1 and ATase2 were purified from stable-transfected cells by affinity chromatography (ProFound column) and then analyzed by Western blotting. The blot with anti-myc (upper panel) demonstrates the presence of ATase1 and ATase2 in the eluate. The blot with anti-BACE1 (lower panel) shows the presence of endogenous BACE1 in the eluate. The bands visible with the anti-BACE1 antibody correspond to the immature and partially-mature forms of the proteins. The migration of the different forms of BACE1 on a similar SDS-PAGE system is shown for comparison (right panel). In FIG. 6(B), the steady-state levels of endogenous BACE1 in ATase1, ATase2, and ATase1+2 expressing cells were analyzed by immunoblotting of total cell lysates. In FIG. 6(C), Aβ levels in the conditioned media were determined by standard sandwich ELISA. Results are the average (n=3)±S.D. Asterisks indicate statistical significance (*, p<0.05; #, p<0.005). In FIG. 6(D), endogenous BACE1 was immunoprecipitated from ATase1, ATase2, and ATase1+2 expressing cells prior to Western blot analysis with an anti-acetylated lysine antibody. The left blot shows that only the ˜55-kDa band corresponding to immature BACE1 could be visualized with an anti-acetylated lysine antibody. The migration of the different forms of BACE1 on a similar SDS-PAGE system is also shown for comparison (right panel).

FIG. 6(E, F) shows data from H4 (human neuroglioma) cells treated with two different siRNA targeting ATase1 prior to real-time quantitation of ATase1 mRNA levels (E) and Western blot analysis of BACE1 steady-state levels. In FIG. 6(F), as control, cells were also treated with scrambled (non-silencing) siRNA. FIG. 6(G-H) shows data from H4 cells that were treated with siRNA targeting ATase2 prior to real-time quantitation of ATase1 mRNA levels (G) and Western blot analysis of BACE1 steady-state levels (H). As control, cells were also treated with scrambled (non-silencing) siRNA. Symbols indicate statistical significance (*, p<0.05; #, p<0.005). FIG. 6(I) shows Aβ levels in the conditioned media of siRNA-treated cells were determined by standard sandwich ELISA as in (C). Results are expressed as percent of control (no treatment) and are the average (n=6)+S.D. Symbols indicate statistical significance (*, p<0.05; #, p<0.005).

FIG. 6A (lower panel) clearly shows that BACE1 was present in the elution fraction of the anti-myc column indicating that both acetyltransferases interact with endogenous BACE1 in vivo. Interestingly, only the immature and partially-mature forms of BACE1 could be immunopurified with ATase1 and ATase2 (FIG. 6A; compare the lower panel with the right one showing the migration pattern of BACE1 following immunopurification with a similar system). These results are perfectly consistent with the fact that ATase1 and ATase2 are localized in the ER and ERGIC system (FIGS. 2C and 4C), where both the immature and partially-mature forms of nascent BACE1 can be found. Therefore, when taken together, these results indicate that ATase1 and ATase2 interact with nascent BACE1 in the ER/ERGIC system.

Lysine acetylation of nascent BACE1 results in molecular stabilization and increased steady-state levels of the β secretase. The overexpression of ATase1 and ATase2 affected the levels of BACE1 and/or the production of Aβ. The results shown in FIG. 6A indicate that both ATase1 and ATase2 caused an increase in the steady-state levels of endogenous BACE1, with ATase2 displaying an apparent stronger effect, which could be related to the more powerful acetylCoA:lysine acetyltransferase activity observed in vitro (compare FIG. 4B to FIG. 2B for total extracts and FIG. 4D to FIG. 2E for the individual fractions). These results were supported by the increased levels of Aβ secreted in the conditioned media (FIG. 6C) and by the apparent increase in the lysine acetylation of endogenous BACE1 (FIG. 6D) in ATase1 and ATase2 expressing cells.

Late-Onset AD Patients Display Increased Levels of ATase1 and ATase2

Ceramide treatment of different cell lines results in increased acetylation and stabilization of BACE1 (Costantini et al., 2005, Biochem. J. 391: 59-67). The increased activation of ceramide in the brain caused by either normal aging or by the hyperactivation of IGF1-R signaling in p44^{+/+} transgenic mice, a model of accelerated aging, results in increased acetylation and stabilization of BACE1. Therefore, the effects induced by ceramide on the mRNA levels of ATase1 and ATase2 in H4 (human neuroglioma) and SH-SY5Y (human neuroblastoma) cells were analyzed. CHO cells were not analyzed because only the human sequence of ATase1 and ATase2 is currently known.

Brain tissue from late-onset AD patients, who are known to have higher levels of ceramide, was also analyzed. For this purpose, quantitative real-time PCR was employed, and to compare the mRNA levels detected in the frontal lobe of four AD patients (average age: 88; age range: 83-93) and four age-matched controls (average age: 88; age range: 85-91).

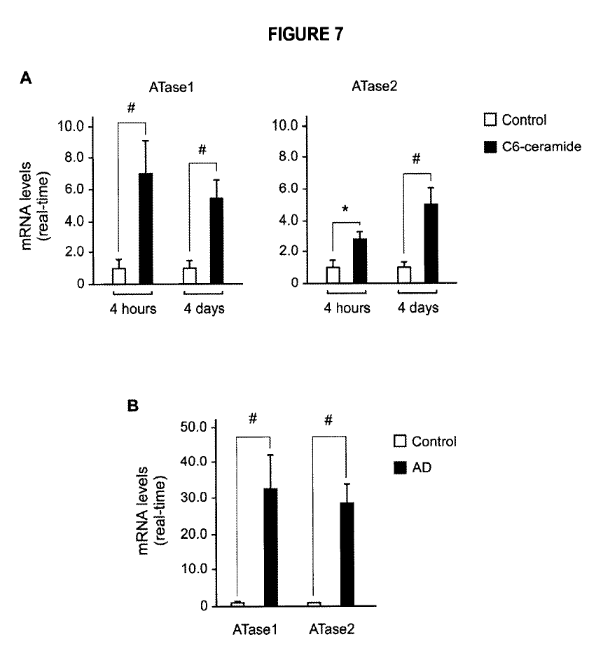

FIG. 7 illustrates how ATase1 and ATase2 are upregulated in AD brains and following ceramide treatment. FIG. 7(A) shows SH-SY5Y cells were treated with C6-ceramide (10 μM) for the indicated period of time prior to real-time quantitative PCR. The 4-hour treatment was performed in the presence of 0.5% fetal bovine serum whereas the 4-days treatment was performed in the presence of 10% fetal bovine serum, as previously described (Puglielli et al., 2003, J. Biol. Chem. 278: 19777-19783; Costantini et al., 2007, Biochem. J. 407: 383-395)). Results are expressed as fold ±S.D. of control (no treatment). Asterisks indicate statistical significance (*, p<0.05; #, p<0.005). No difference was observed in the mRNA levels of GAPDH, which was used as internal control. FIG. 7(B) A is a cDNA library of late-onset AD (n=4 different subjects) and age-matched controls (n=4 different subjects) was analyzed by quantitative real-time PCR. Results are expressed as fold ±S.D. of age-matched controls. No difference was observed in the mRNA levels of GAPDH, which was used as internal control.

FIG. 7B shows a dramatic difference with AD patients displaying a ˜30-fold increase in the mRNA levels of both ATase1 and ATase2. The analysis was repeated three times to confirm the very high levels observed in AD patients, consistently obtaining the same results.

Under basal conditions the mRNA levels of both ATases were higher in SH-SY5Y than in H4 cells, suggesting possible cell-type differences. However, when treated with ceramide, both cell lines displayed a marked activation of transcription of ATase1 and ATase2, as detected by quantitative real-time PCR (FIG. 7A). The effect could be observed as early as 4 hours after treatment and was still evident after 4 days, which is consistent with the time-course that was earlier described (Puglielli et al., 2003, J. Biol. Chem. 278: 19777-19783). At early-time points ATase1 displayed a more dramatic response to ceramide than ATase2, which could be due to the fact that the baseline levels of ATase2 were consistently found higher than ATase1.

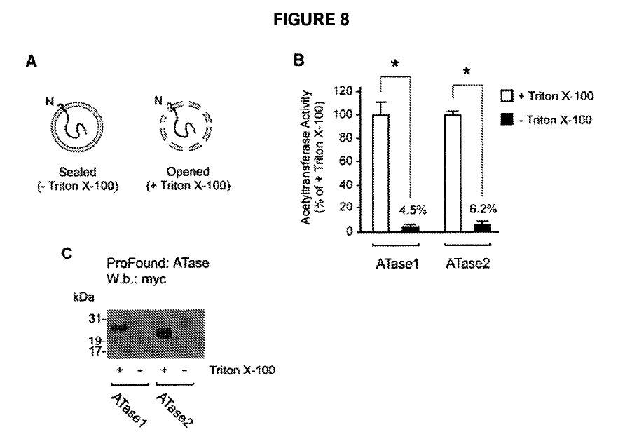

The hydrophilic/hydrophobic plot (FIG. 1B) together with the apparent topology of ATase1 and ATase2 predicts the C-terminal domain, which contains the acetylCoA:lysine acetyltransferase homology domain, to face the lumen of the ER/ERGIC system. To determine that this is indeed the case, in vitro assays were conducted of acetylCoA:lysine acetyltransferase activity recovered from ERGIC vesicles of ATase1 or ATase2 expressing cells under two different experimental conditions: in the presence or absence of mild concentrations (0.2-0.5%) of Triton X-100. FIG. 8 illustrates that the C-terminal and catalytically active domain of ATase1 and ATase2 faces the luminal site of the ER/ERGIC system. FIG. 8(A) is a schematic view of the rationality of the experiments reported here. The N-terminus of either ATase1 or ATase2 is indicated. The C-terminal attached myc-tag is in red. FIG. 8(B) shows how ER and ERGIC vesicles from ATase1 and ATase2 expressing cells were incubated with affinity purified BACE1 and [³H]AcetylCoA for 1 hour at 30° C. Reaction was stopped by lowering the temperature; BACE1 was then purified again and analyzed on a scintillation liquid counter. Vesicles were used under the “sealed” (no detergent; black bars) or “opened” (+ detergent; white bars) condition. Results are expressed as percent of BACE1 acetylation obtained in the presence of detergent (opened condition) and are the average (n=6)±S.D. Numbers on top of the black bars indicate the percent of total acetylCoA:lysine acetyltransferase activity recovered in the absence of detergent (sealed condition). FIG. 8(C) shows how sealed and opened vesicles shown in (A) were incubated with an anti-myc antibody covalently attached to aldehyde-activated agarose beads (ProFound system) for immunoprecipitation. After extensive washing, bound proteins were eluted and analyzed by SDS-PAGE and immunoblotting.

When prepared, vesicles are sealed and of the same membrane topographical orientation as in vivo. Therefore, under normal conditions (“sealed”; FIG. 8A) the acetylation of affinity purified BACE1 can occur only if the catalytic site of the enzyme (ATase1 or ATase2) is facing the outside/cytosolic face of the vesicles. Conversely, if the catalytic site of the enzyme resides in the luminal face of the vesicles, the reaction will only occur in the presence of mild concentrations of detergent, which will allow access of both the acceptor (purified BACE1) and the donor (radiolabeled acetylCoA) to ATase1 or ATase2 (“opened”; FIG. 8A). FIG. 6B clearly shows that the acetyltransferase activity of native ATase1 and ATase2 could only be observed in the presence of Triton X-100, indicating that the catalytic site is facing the lumen of the organelle in its native conditions. The activity recovered in the absence of Triton X-100 was in the normal range of latency (3-5%) observed with glucose-6-phosphatase sialyl-transferase methods.

Both ATase1 and ATase2 have a myc-tag at the C-terminus, which is predicted to face the lumen of the ER/ERGIC system. Therefore, the above vesicles were incubated with an anti-myc antibody covalently attached to aldehyde-activated agarose beads (ProFound system) for immunoprecipitation. The experiment was performed under both the “opened” and “sealed” conditions (FIG. 8A) to determine the topology of the enzymes. FIG. 8C shows that ATase1 and ATase2 could be immunoprecipitated only when the vesicles were used under the “opened” condition, which allowed the anti-myc antibody to interact with C-terminal myc-tag. These results indicate that both ATase1 and ATase2 have the predicted topology, with the catalytic C-terminal domain facing the lumen of the ER/ERGIC system, where the lysine acetylation of nascent BACE1 normally occurs.

Taken together, the above results clearly indicate that both ATase1 and ATase2 are upregulated by ceramide treatment and that AD patients, who display increased levels of ceramide in the neocortex, have increased levels of ATase1 and ATase2. Therefore, biochemical characterization of the acetylation/deacetylation machinery might lead to the identification of novel pharmacological strategies for the prevention of late-onset AD.

The biochemical analysis of ATase1 and ATase2 indicates that they can act as independent enzymes in vitro supporting the idea that they are not part of a catalytic complex where they simply act as co-factors of the reaction. Even though they are 88% identical at the amino acid level and share several biochemical features, ATase1 and ATase2 seem to display two important differences. First, ATase1 could recognize and acetylate the fully-mature form of BACE1—at least in vitro—whereas ATase2 could not; second, ATase1 could be detected with an anti-acetylated lysine antibody whereas ATase2 could not. The former might indicate differences in substrate recognition properties of the two enzymes whereas the latter might suggest different catalytic mechanisms. These differences might become important when attempting to design biochemical inhibitors for pharmacological purposes (Hodawadekar and Marmorstein, 2007, Oncogene 26: 5528-5540). For example, it is important to point out that p300 HAT contains an “activation/regulatory loop” that undergoes autoacetylation and functions as an allosteric switch that regulates its own acetyltransferase activity: when hypoacetylated it inhibits, while when autoacetylated it activates the activity of the enzyme.

Subcellular Distribution Profile of BACE1, ATase1, and ATase2

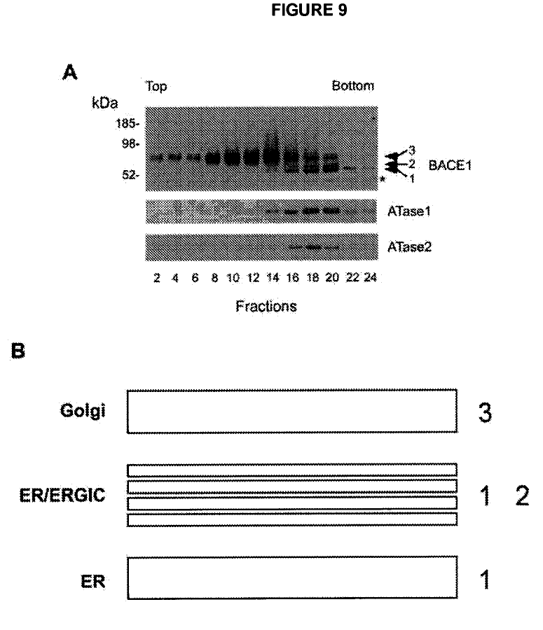

FIG. 9(A) shows the subcellular distribution profile of BACE1, ATase1, and ATase2 was analyzed as described in FIGS. 2C and 4C. The three species corresponding to the mature and the biosynthetic intermediates of BACE1 are indicated by numbers. The asterisk (*) points to a non-specific band. The typical migration of the subcellular markers in a similar gradient is shown in FIGS. 2C and 4C. The figure compares the distribution profile of ATase1 (also shown in FIG. 2C) and ATase2 (also shown in FIG. 4C) to that of BACE1. Note that the subcellular distribution profile of the two biosynthetic intermediates of BACE1 (labeled with 1 and 2) that co-immunoprecipitate with ATase1 and ATase2 in FIG. 6A overlaps with both acetyltransferases.

FIG. 9 (B) shows the schematic view of the subcellular distribution of the different BACE1 forms. Form 1 has an apparent molecular mass of 52-55-kDa and migrates as the immature nascent form of BACE1 on SDS-PAGE. It is found in both the ER and ERGIC but not in the Golgi apparatus. This form is EndoH sensitive indicating that it has already received the Asn-linked incomplete oligosaccharide. Form 2 displays a molecular mass of ˜65-kDa on SDS-PAGE and is only found in the ERGIC. Not wanting to be bound by the following theory, it most likely represents an intermediate biosynthetic species of nascent BACE1. Whether the different migration on gel electrophoresis is caused by the lysine acetylation, differential glycosylation, or another—not characterized—modification is unknown. Both form 1 and form 2 are acetylated (see FIG. 6A), can be co-immunoprecipitated with ATase1 and ATase2 (see FIG. 6A), and are present in the same compartments that contain ATase1 and ATase2. Form 3 migrates as the fully-glycosylated species of BACE1, is not acetylated in vivo, does not immunoprecipitate with ATase1 and ATase2 (see FIG. 6A), and is found in the Golgi apparatus. It corresponds to the mature form of BACE1, which originates after complete maturation of the oligosaccharide chain and deacetylation in the Golgi apparatus. The term “nascent” or “immature” is used to indicate the ER— and ERGIC-based forms of BACE1 that have not yet completed glycosylation in the Golgi apparatus. Similarly, the term “mature” is used to indicate the BACE1 form that arises from the completion of glycosylation in the Golgi apparatus. For the apparent migration (and molecular mass) of the different forms described here, see also FIGS. 3B, 5A, and 6A.

It is to be understood that this invention is not limited to the particular devices, methodology, protocols, subjects, or reagents described, and as such may vary. It is also to be understood that the terminology used herein is for the purpose of describing particular embodiments only, and is not intended to limit the scope of the present invention, which is limited only by the claims. Other suitable modifications and adaptations of a variety of conditions and parameters, obvious to those skilled in the art of biochemistry and molecular biology, are within the scope of this invention. All publications, patents, and patent applications cited herein are incorporated by reference in their entirety for all purposes.

What is claimed is:

1. An in vitro method, comprising:
a) reacting a polypeptide at least 87% identical to the amino acid sequence of SEQ ID NO:1 or SEQ ID NO:2, a substrate, an ER-derived vesicle, an acetylCoA, and
b) measuring the acetyltransferase activity of the polypeptide, wherein the acetyltransferase activity comprises acetylation of the substrate.

2. The method of claim 1 wherein the polypeptide is at least 95% identical to the amino acid sequence of SEQ ID NO:1.

3. The method of claim 1 wherein the polypeptide is at least 95% identical to the amino acid sequence of SEQ ID NO:2.

4. The method of claim 1 comprising measuring the acetylCoA:lysine acetyltransferase activity of the polypeptide.

5. The method of claim 1 wherein the substrate is BACE1.

6. The method of claim 1 wherein the acetylation comprises acetylation of one or more lysine residues of the substrate.

7. The method of claim 1 wherein the acetyltransferase activity comprises acetylation of the substrate in the ER-derived vesicle.

8. An in vitro method for identification of a candidate compound as a compound that may be useful for the treatment of Alzheimer's disease, said method comprising the steps of:
a) providing a polypeptide at least 87% identical to the amino acid sequence of SEQ ID NO:1 or SEQ ID NO:2,
b) contacting the polypeptide with the candidate compound, and
c) measuring the acetyltransferase activity of the polypeptide, wherein a decrease in the acetyltransferase activity of the polypeptide, relative to the acetyltransferase activity of the polypeptide not contacted with the candidate compound, identifies the candidate compound as a compound that may be useful for the treatment of Alzheimer's disease.

9. The method of claim 8 wherein the polypeptide acetylates an aspartic peptidase in the ER.

10. The method of claim 9 wherein the aspartic peptidase is BACE1.

11. The method of claim 8 wherein the polypeptide is ATase1.

12. The method of claim 8 wherein the polypeptide is ATase2.

13. The method of claim 8 further comprising measuring the acetylCoA:lysine acetyltransferase activity of the polypeptide.

14. The method of claim 8 wherein the polypeptide is expressed in a cell.

15. An in vitro method, comprising:
a) reacting an ATase1 or ATase2 with a known concentration of a substrate and a known concentration of a test compound, and
b) measuring the acetyltransferase activity of the ATase1 or ATase2, wherein a decrease in the acetyltransferase activity of the ATase1 or ATase2, relative to the acetyltransferase activity of the ATase1 or ATase2 in the absence of the test compound, identifies the test compound as a compound that may be useful for the inhibition of acetyltransferase activity of the ATase1 or ATase2.

16. The method of claim 15 wherein the substrate is BACE1.

17. The method of claim 15 further comprising measuring the acetylCoA:lysine acetyltransferase activity of the ATase1 or ATase2.

18. The method of claim 15 further comprising measuring the acetyltransferase activity of the ATase1 or ATase2 in an endoplasmic reticulum.

19. An isolated or recombinantly produced polypeptide having an amino acid sequence at least 87% identical with an amino acid sequence selected from a group consisting of the amino acid sequence of SEQ ID NO:1 and SEQ ID NO:2, wherein the polypeptide is capable of acetylating a substrate.

20. The polypeptide of claim 19, which is capable of acetylating a substrate in an endoplasmic reticulum.

21. The polypeptide of claim 19, which has an acetylCoA:lysine acetyltransferase activity.

22. The polypeptide of claim 19, wherein the polypeptide is capable of acetylating BACE1.

23. A method, comprising acetylating a substrate using an isolated or recombinantly produced polypeptide having an amino acid sequence at least 87% identical with an amino acid sequence selected from a group consisting of the amino acid sequences of SEQ ID NO:1 and SEQ ID NO:2.

24. The method of claim 23 wherein the substrate is BACE1.

25. A system, comprising:
a) an isolated polypeptide having an amino acid sequence at least 87% identical with an amino acid sequence selected from a group consisting of the amino acid sequences of SEQ ID NO:1 and SEQ ID NO:2,
b) an acetyl donor, and
c) a substrate, wherein the polypeptide has an acetyltransferase activity for acetylating the substrate.

26. The system of claim 25, wherein the polypeptide has an acetylCoA:lysine acetyltransferase activity

27. The system of claim 25 wherein the acetyl donor is acetyl coenzyme A.

28. The system of claim 25 wherein the substrate is BACE1.
